(19) United States
(12) Patent Application Publication
(10) Pub. No.: US 2025/0169701 A1
(43) Pub. Date: May 29, 2025

(54) Biometric Sensor Infused Garment

(71) Applicant: Pivot Sports Global LLC, Friday Harbor, WA (US)

(72) Inventor: John C. Lally, Friday Harbor, WA (US)

(73) Assignee: Pivot Sports Global LLC, Friday Harbor, WA (US)

(21) Appl. No.: 18/709,025

(22) PCT Filed: Nov. 9, 2022
(86) PCT No.: PCT/US22/79588

§ 371 Date: May 10, 2024

Related U.S. Application Data
(60) Provisional application No. 63/263,858, filed on Nov. 10, 2021.

(51) Int. Cl. A61B 5/0205; A61B 5/00

(52) U.S. Cl. CPC A61B 5/0205; A61B 5/6805

(57) ABSTRACT

A biometric wearable device includes a garment arranged for wear about a torso of a subject, a biometric sensor arranged to detect the heart-caused signals of the subject when the subject is wearing the garment, a band integrated at a heart-active region of the garment and arranged to contain the biometric sensor, and at least one force amplification structure positionable in a force amplification receptacle of the band. The at least one force amplification structure is arranged to provide increased directional pressure to the biometric sensor. In some cases, the garment is a T-shirt or other like article, and the heart-active region is proximate an area of the subject's torso where the heart-caused signals are detectable.

### BACKGROUND

### Technical Field

The present disclosure generally relates to biometric sensors. More particularly, but not exclusively, the present disclosure relates to a garment having at least one biometric sensor and certain other structure arranged to desirably position said sensor or sensors about a selected area of the body wearing the garment.

### Description of the Related Art

Some aspects of wearable biometric sensor devices having structure to position the sensor or sensors about the chest of a user, which may be useful in understanding the background of the present disclosure, are described in the following publications:

U.S. Pat. No. 3,268,845 to Whitmore, first filed September 1965, teaches an apparatus for detecting subjective respiration and movement using an electromechanical transducer to recognize chest movements with minimum discomfort to the subject.

U.S. Pat. No. 4,889,131 to Salem et. al, first filed December 1988, covers a portable belt arranged to: 1) monitor heart and breathing functions (i.e., EKG sensors and a respiration sensor); 2) detect alarm conditions; and 3) transmit them to a remote receiver. Salem's respiration sensor is configured to detect tension changes in the belt due to breathing, and the belt can include EKG sensors. Hence, Salem may be understood to teach a belt structure that tightly encircles the user's chest.

U.S. Pat. No. 6,986,771 to Paul et. al., first filed May 23, 2003, describes a spine stiffening and stabilization system having interlocking flexible elements that can be secured in a rigid configuration.

U.S. Pat. Appl. No. 2003/0214408 to Grajales et al., first filed May 2002, teaches a type of wearable garment sensor composite data formed by analyzing data from two or more sensors sensing the same parameter at the same time.

U.S. Pat. No. 7,680,523 to Rytky, first filed December 2004, added a garment having a heart rate monitor held in place by a flexible film to the state of the art. The flexible film structure of Rytky comprises a first insulation layer and at least one electric conductor layer formed on top of the first insulation layer. An electrode area of the flexible film is configured to establish an electric contact between an electrocardiogram sensor and the surface of the user's skin.

U.S. Pat. No. 7,970,451 to Hassonjee et al., first filed March 2005, added textile-based electrodes to the state of the art. Hassonjee's electrodes include a fabric portion having stretch-recovery, non-conductive yams and an electrically conductive region having stretch-recovery, electrically conductive yam filaments. The electrodes can further include float yarns and can be configured in a textured or ribbed construction. When incorporated into a garment, the electrodes can be used to monitor biophysical characteristics, such as the garment wearer's heart rate.

The family of patents including U.S. Pat. Nos. 8,668,653; 9,026,200; 9,414,785; 9,433,379 to Nagata et al., first filed March 2005, and these patents added, in various embodiments, integrating several sensors instead of using a single sensor into a garment to account for the different physical characteristics of each user. Real-time analysis determines which sensor or sensors provide a strongest signal, and data from the selected sensor or sensors is collected for analysis.

U.S. Pat. Appl. No. 2009/0149727 to Truitt et al., first filed December 2007, added to the state of the art a teaching of a biometric sensor device arranged to collect data from a non-human subject.

U.S. Pat. Appl. No. 2009/0306485 to Bell et al., first filed June 2008, added to the state of the art a ‘kit’ having modular sensors and a set of flexible conductors of many shapes and lengths such that a customized system can be formed for any particular user having any particular shape, size, etc.

U.S. Pat. No. 8,467,861 to Rytky, first filed April 2009, added to the state of the art a mechanism to attach electrocardiogram sensors to a wearable garment such that an active portion of each sensor is in contact with the user's skin. An attachment portion (e.g., a ‘post’) extends through the clothing or band, and a detector portion (e.g., an ‘anchor’) couples the sensors and the detector portion together.

U.S. Pat. Nos. 8,475,371; and 9,801,583, to Derchak et al., first filed September 2009, added a garment with a circularly formed magnetometer arranged to detect deformative motion caused by a user of the garment engaging in physical activity to the state of the art.

U.S. Pat. No. 9,028,404 to DeRemer et al., first filed July 2010, added to the state of the art a teaching of a sensor subsystem “dock” integrated in a stretchable band of a wearable garment. The sensor subsystem dock is arranged to collect heart rate data and further arranged to receive a removable communications module, which module is used to transmit the collected data.

U.S. Pat. No. 8,585,606 to McDonald et al., first filed September 2010, added a known garment with an integrated band and physiological sensors to the state of the art. The subject matter added by McDonald includes complex software to determine the user's real-time biomechanical load, which is used to further understand the user's physiological performance.

U.S. Pat. Appl. No. 2012/0158074 to Hall et al., first filed December 2010, added to the state of the art with teaching of a washable/cleanable shirt having physiological sensors and defibrillation circuitry integrated therein.

U.S. Pat. No. 10,610,118 to Jaakkola et al., first filed January 2011, added to the state of the art a teaching of water-tight physiological sensors permanently integrated with a textile garment.

A family of patents including U.S. Pat. Nos. 8,948,839; 9,282,893; 10,159,440; 10,462,898; U.S. Pat. No. 10,736,213 to Longinotti-Buitoni et al., first filed September 2012, added to the state of the art various embodiments of compression fabric garments impregnated with stretchable, conductive ink. The conductive ink is used in a creative design visible in the garment to electrically connect electronic components integrated in a garment.

U.S. Pat. Appl. No. 2016/0038083 to Ding et al., first filed August 2014, added to the state of the art a teaching of flexible, conductive fabric used as a dynamic sensor that collects data based on a change in resistance of the conductor as the fabric stretches or otherwise changes length.

U.S. Pat. No. 9,566,033 to Bogdanovich et al., first filed in November 2014, added to the state of the art a teaching of a wireless protocol (e.g., BLUETOOTH SMART) to offload data from a sensor-laden wearable garment.

U.S. Pat. No. 10,959,467 to, Nakao first filed March 2016, added to the state of the art with teaching of a stretchable, conductive fabric formed in a particular way (i.e., conductive particles dispersed at a particular concentration in a non-crosslinked elastomer) such that the conductors have a particular width/thickness ratio and a particular durability (e.g., maintaining electrical continuity despite repeating 20% elongation for ten (10) or more times).

U.S. Pat. Appl. No. 2018/0014780 to Sotzing et al., first filed July 2016, added to the state of the art with teaching of improved flexible sensors having a particular chemical composition.

U.S. Pat. Appl. No. 2021/0142894 to Raisanen, first filed April 2017, added to the state of the art a teaching of a system that includes both wearable sensors and bed-based sensors for around-the-clock sensor monitoring.

U.S. Pat. No. 10,076,462 to Johnson et al., first filed April 2017, added to the state of the art a teaching of a motor-driven compression mechanism.

U.S. Pat. Appl. No. 2020/0128670 to Chong-Rodriguez et al., first filed May 2017, added to the state of the art a teaching of biometric sensors for use in a wearable garment, wherein the sensors include padding layers around sealed electronic circuitry.

U.S. Pat. Appl. No. US 2018/0368495 to Simmons, first filed June 2017, added to the state of the art a teaching of a sports bra having integrated sensors.

U.S. Pat. Appl. No. 2019/0133215 to Whalen, first filed July 2017, added to the state of the art a teaching of a garment expressly structured via tubing and compressed air to restrict blood flow of an underlying muscle or group of muscles.

U.S. Pat. Appl. No. 2019/0336038 to Gorgutsa et al., first filed May 2018, added to the state of the art with teaching of a wearable garment having integrated sensors and a dipole antenna.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which, in and of itself, may also be inventive.

### BRIEF SUMMARY

The following is a summary of the present disclosure to provide an introductory understanding of some features and context. This summary is not intended to identify key or critical elements of the present disclosure or to delineate the scope of the disclosure. This summary presents certain concepts of the present disclosure in a simplified form as a prelude to the more detailed description that is later presented.

The device, method, and system embodiments described in this disclosure (i.e., the teachings of this disclosure) include certain force amplification structures deployed in a garment to enable the collection of improved quality heart-caused electrical signal data of a subject wearing the garment. As described herein, such force amplification structures create directional pressure on one or more biometric sensors to facilitate the collection of improved quality data without reducing comfort of the garment as in the case of high-compression garments, bands, harnesses, and the like.

In a first embodiment, a biometric wearable device includes a garment such as a clothing article arranged for wear about a torso of a subject, a biometric sensor arranged to detect the heart-caused signals of the subject when the subject is wearing the garment, a band positioned at a heart-active region of the garment and arranged to contain the biometric sensor, and at least one force amplification structure positioned in a force amplification receptacle of the band. The at least one force amplification structure is arranged to provide increased directional pressure to the biometric sensor. The garment in some cases includes a first opening for substantially encircling a neck area of the subject that is wearing the garment, a second opening for substantially encircling a right extremity of the subject, a third opening for substantially encircling a left extremity of the subject, a fourth opening for substantially encircling a lower portion of the torso of the subject, and the heart-active region which, when the subject is wearing the garment, is proximate an area of the torso of the subject where heart-caused signals are detectable.

In some cases of the first embodiment, the band is arranged to position an active portion of the at least one biometric sensor in direct contact with the torso of the subject. In some cases of the first embodiment, the biometric sensor includes at least two electrodes. Sometimes, the band is at least ten inches (10 in.) long, and sometimes, the band is a continuous band having at diameter of between about eight to fifteen inches (8 in. to 15 in.). In these and other cases, the band is between about one to four inches (1 in. to 4 in.) wide, and these and other cases, the band is between about one to five hundred mils (0.001 in. to 0.5 in.) thick.

In some cases of the first embodiment, the band is formed from a material having elastic properties. And sometimes, the band is formed from a first material having stronger elastic properties than a second material used to form the garment. That is, both the band and the garment are formed from material having elastic properties, but the elastic properties of the band are stronger than the electric properties of the garment.

In some cases of the first embodiment, the band exposes at least one electromechanical structure arranged to pass signal information associated with the at least one biometric sensor, and in at least some of these cases, the at least one electromechanical structure is a wire or an electrical connector.

In some cases, the garment is a shirt. In some cases, the subject is a human being, but in other cases, the subject is a non-human mammal.

And in some cases of the first embodiment, the biometric sensor is one of a plurality of biometric sensors integrated in the band.

In a second embodiment, a method includes: providing a garment with a band, the band having a biometric sensor, the band further having a force amplification receptacle; positioning at least one force amplification structure in the force amplification receptacle of the band, wherein the at least one force amplification structure is arranged to provide increased directional pressure to the biometric sensor; and detecting, with the biometric sensor, at least one heart-caused signal of a subject wearing the garment.

In some cases of the second embodiment, the method also includes the act of detecting, with the biometric sensor, a plurality of heart-caused signals of the subject wearing the garment over a determined period of time. In these and other cases, the method includes communicating the at least one heart-caused signal to a remote computing device. And sometimes, the method also includes improving quality of the detected at least one heart-caused signal by adding at least one additional force amplification structure to the force amplification receptacle or removing one of the at least one force amplification structures from the force amplification receptacle.

In a third embodiment, a system includes: a garment arranged for wear by a subject, the garment including a heart-active region which, when the garment is worn by the subject, is proximate an area of the subject where heart-caused signals are detectable, and a biometric sensor arranged to detect the heart-caused signals of the subject when the subject is wearing the garment. The system also includes a band positioned at least in part at the heart-active region of the garment and arranged to contain the biometric sensor; at least one force amplification structure positioned in a force amplification receptacle of the band, wherein the at least one force amplification structure is arranged to provide increased directional pressure to the biometric sensor; and at least one remote computing device arranged to receive information communicated from the biometric sensor, said information representative of the detected heart-caused signals.

This Brief Summary has been provided to describe certain concepts in a simplified form that are further described in more detail in the Detailed Description. The Brief Summary does not limit the scope of the claimed subject matter, but rather the words of the claims themselves determine the scope of the claimed subject matter.

### BRIEF DESCRIPTION OF THE SEVERAL VIEWS OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIG. 1 is an embodiment of human anatomy illustrating a heart-active region proximate an area of the human's torso;

FIGS. 2A-2G are various embodiments of a biometric sensor infused garment;

FIGS. 3A and 3B are embodiments of a band structure useful in one or more embodiments of a biometric infused garment;

FIG. 4 is a system embodiment deploying a band structure arranged to contain at least one biometric sensor; and

FIG. 5 is a data flow diagram illustrating a non-limiting process that may be used by embodiments of a band structure system such as the one illustrated in FIG. 4.

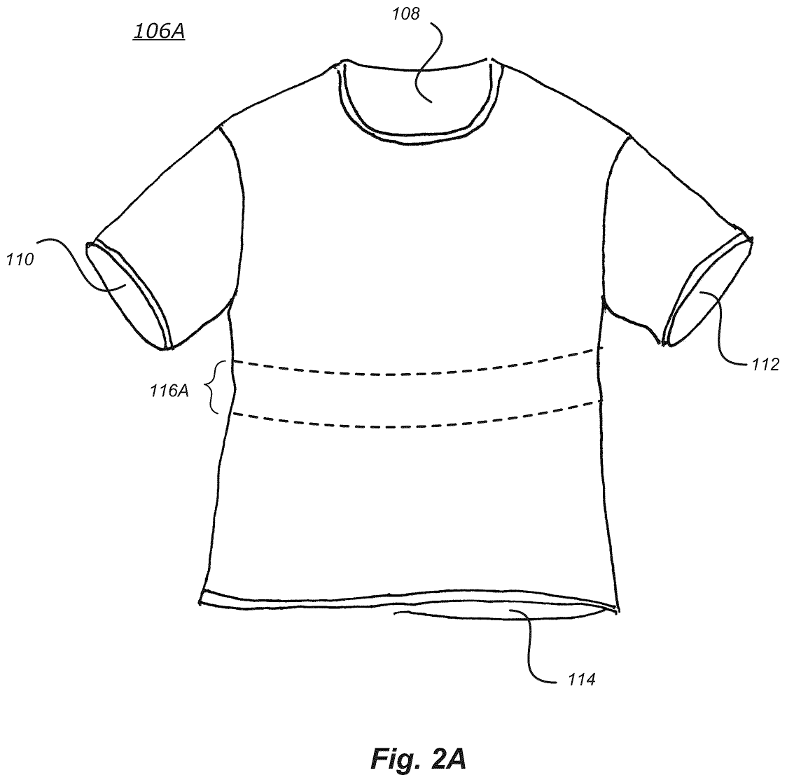

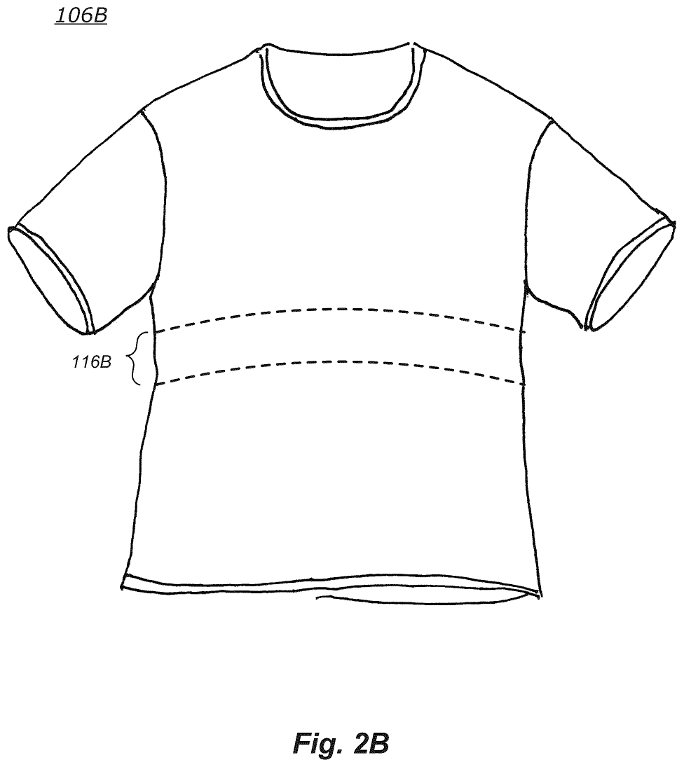

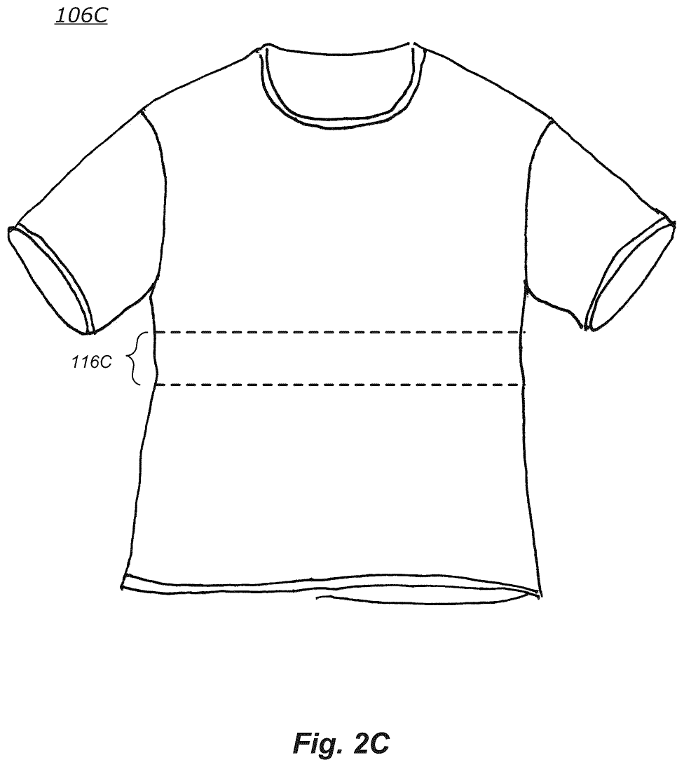

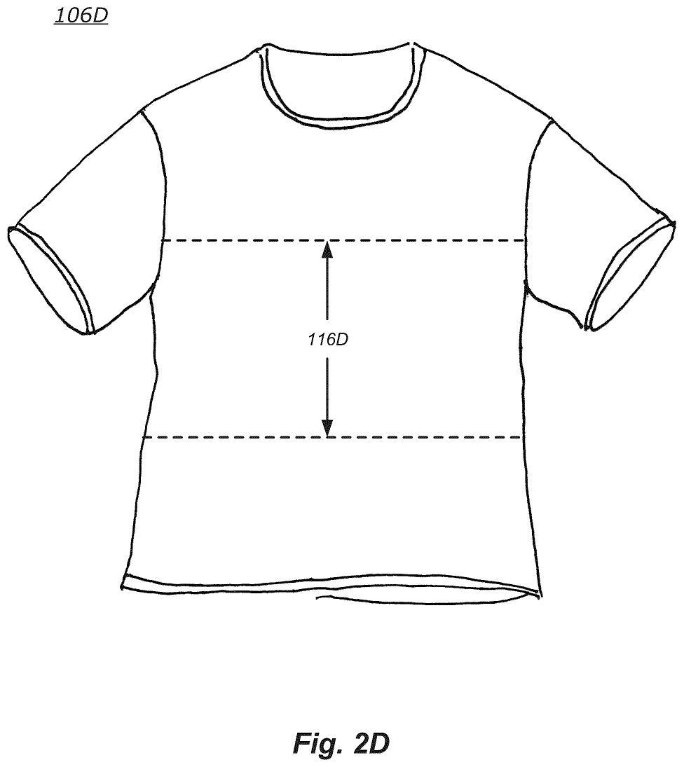

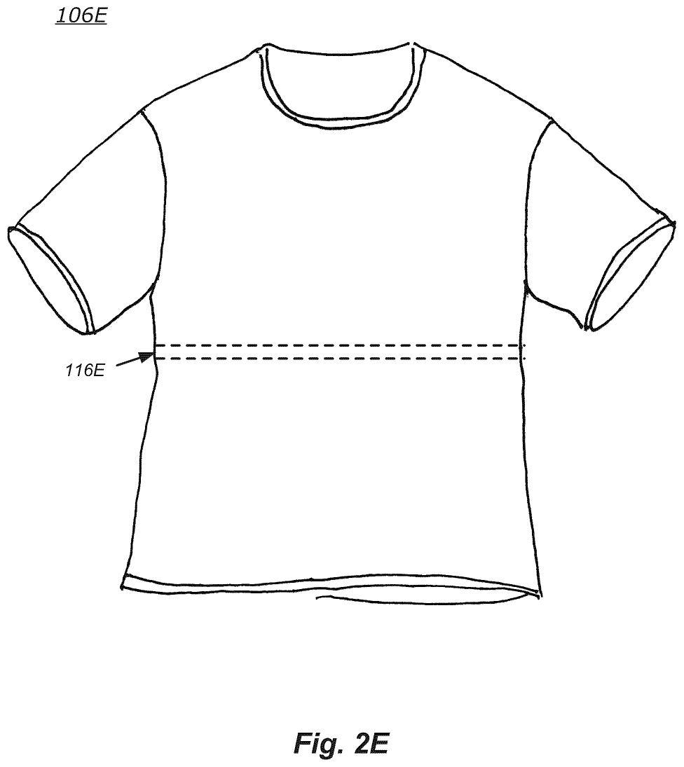

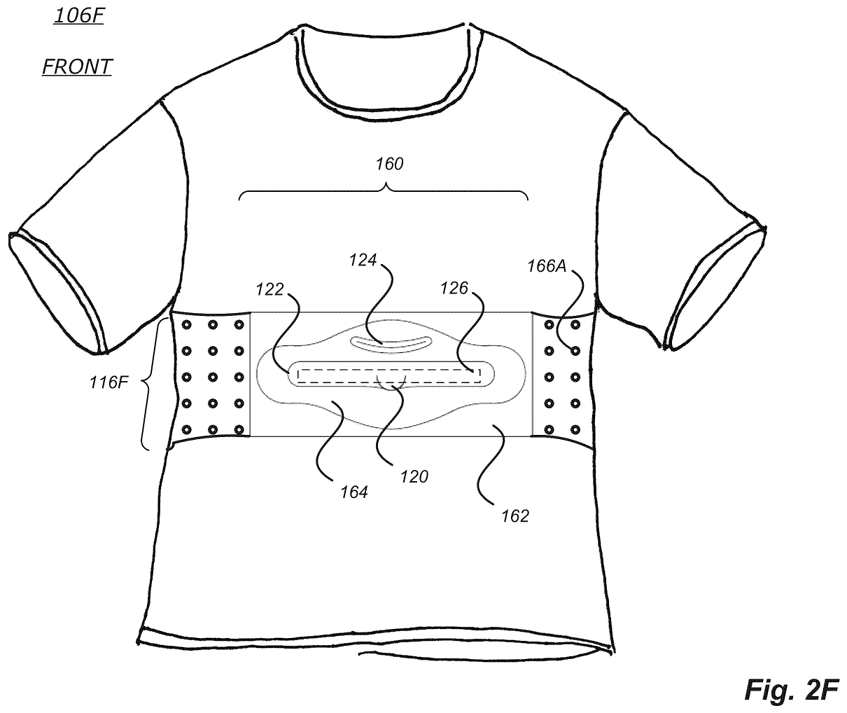

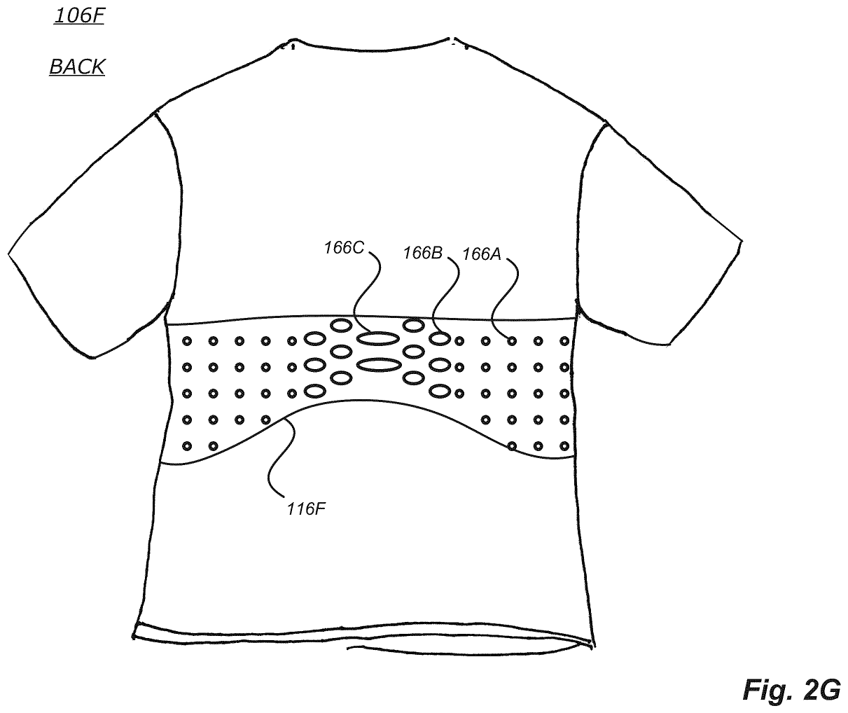

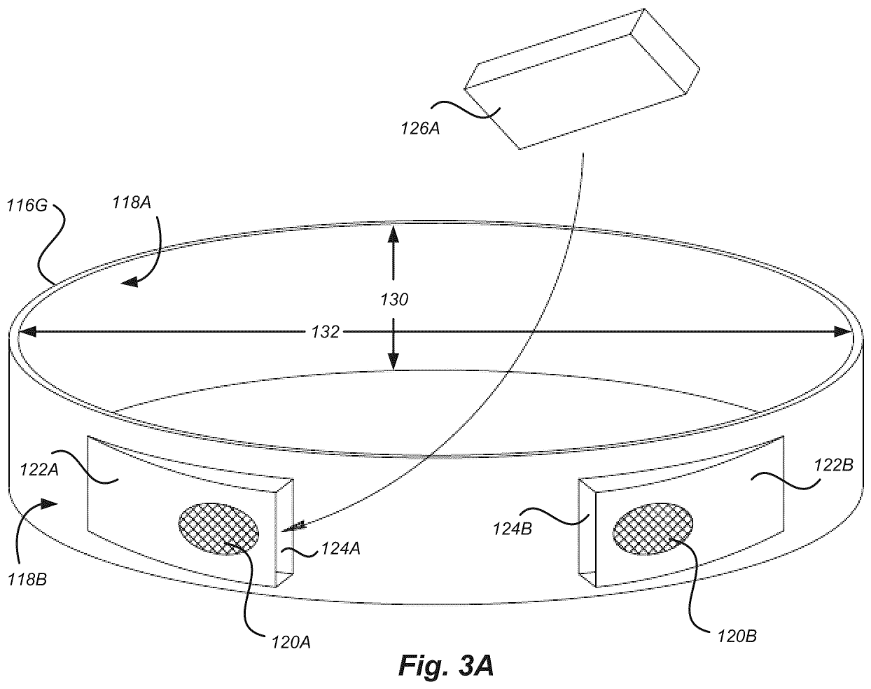

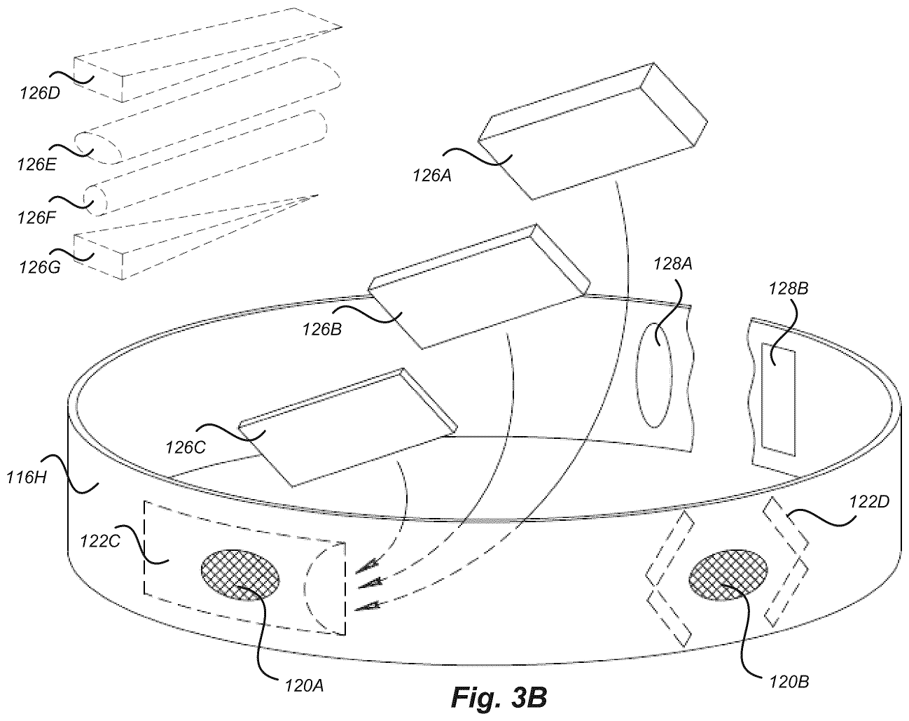

In the present disclosure, for brevity, certain sets of related figures may be referred to as a single, multi-part figure to facilitate a clearer understanding of the illustrated subject matter. For example, FIGS. 2A-2G may be individually or collectively referred to as FIG. 2. And likewise, FIGS. 3A-3B may be individually or collectively referred to as FIG. 3. Structures earlier identified may not be repeated for brevity.

### DETAILED DESCRIPTION

The present disclosure may be understood more readily by reference to this detailed description and the accompanying figures. The terminology used herein is for the purpose of describing specific embodiments only and is not limiting to the claims unless a court or accepted body of competent jurisdiction determines that such terminology is limiting. Unless specifically defined in the present disclosure, the terminology used herein is to be given its traditional meaning as known in the relevant art.

In the following description, certain specific details are set forth in order to provide a thorough understanding of various disclosed embodiments. However, one skilled in the relevant art will recognize that embodiments may be practiced without one or more of these specific details, or with other methods, components, materials, and the like. In other instances, certain structures have not been shown or described in detail to avoid unnecessarily obscuring more detailed descriptions of the embodiments. These certain structures may include, but are not limited to one or both of garments and biometric data collection devices (e.g., shirts, jackets, vests, a suspender or pair of suspenders, pants, socks, hats, jewelry, glasses, pads, sporting equipment, and gear), sensors, inspection points, computing devices, circuitry, wired and wireless communications protocols, wired and wireless transceivers, radios, communications ports, geolocation devices, and other such electronic, electrical, electromechanical, mechanical, and data collection means. One skilled in the relevant art will further recognize that in some embodiments, or in portions of some embodiments, well-known structures associated with computing systems, including client and server computing systems as well as networks, have not been shown or described in detail to avoid unnecessarily obscuring more detailed descriptions of the embodiments.

Prior to setting forth the embodiments, however, it may be helpful to an understanding thereof to first set forth definitions of certain terms that are used hereinafter.

Biometric data, as used herein, is physiological data. Biometric data may be collected by any number (i.e., quantity) and type of sensors. Biometric data may be processed by any number and type of computing system. A non-exhaustive list of exemplary biometric data, as contemplated herein, includes, but is not limited to, heart-caused signals, heart-rate data, heart rate variability (HRV) data, respiration data, temperature data, motion data, vibration data, motion data (e.g., acceleration data, gyroscopic data, and the like), hydration data, oxygen saturation data, blood pressure data, blood-volume data, blood glucose data, galvanic skin response data (e.g., skin conductance data), skeletal positioning data, sound pressure data, electroencephalogram data, photoplethysmography data, electrocardiogram data, and other such data.

A “garment” is a structure or structures containing or otherwise positioning and/or securing one or more biometric sensors proximate a body. A garment may be a wearable structure such as an article of clothing. Non-limiting examples of garments include hats, headbands and other headwear, scarves, shirts, vests, suspenders, jackets, sleeves, jerseys, belts, pants, leggings, shorts, undergarments, socks, shoes or other footwear, bands (e.g., armbands, leg bands, and other band-like structures), gloves, mittens, wristwatches or like structures, jewelry, glasses, protective padding, and other activity-related equipment and gear (e.g., body armor, sporting equipment, pads, breathing apparatus such as tanks, safety or other protective gear, control equipment such as a steering wheel or joystick, comfort structures such as seats, chairs, and beds, and the like).

“Sensor” refers to . . . A biometric sensor includes any one or more sensors arranged to collect, generate, provide, or otherwise process biometric data. can have any suitable size, shape, and material composition. For example, biometric sensors may include cross-sections or outlines that are generally square, rectangular, circular, elliptical, ovular, polygonal, or any other selected shape. Additionally, or alternatively, biometric sensors may be formed of any one or more of plastic, metal, fabric, a composite, an adhesive, and the like.

As discussed in the background section of the present disclosure, wearable biometric sensor devices date back to at least 1965. Many attempts to perfect such sensor devices have been tried, but all have fallen short. The present inventor has recognized that the collection of a user's biometric data is improved when the sensors are in firm, stable contact with the user's body, and the garment is comfortable and easy to use. Hence, the device, method, and system embodiments described in this disclosure (i.e., the teachings of this disclosure) include a garment that reliably and comfortably places one or more biometric sensors in firm, stable contact with the user's body during certain biometric data collection processes.

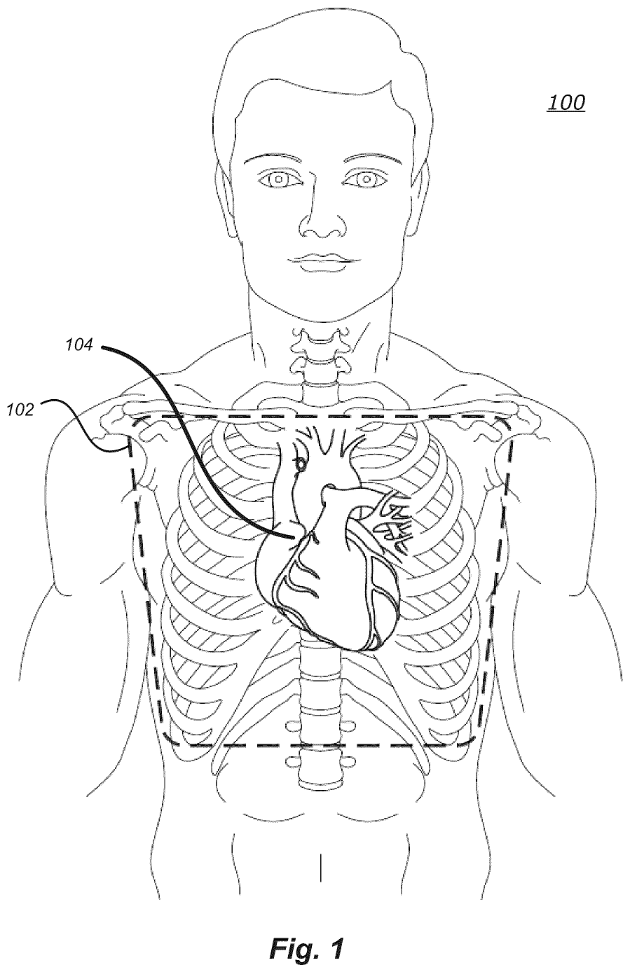

FIG. 1 is an embodiment of human anatomy 100 illustrating a heart-active region 102 proximate a chest area of the human's torso. A representation of the human's heart 104 is shown in FIG. 1. With each heartbeat, an electrical signal (e.g., impulse, energy, voltage, current, wave, excitation, stimulation, or the like) travels through the heart. This signal, which causes the heart muscle to squeeze and pump blood from the heart and through the body, is detectable with certain sensors. The heart-active region of the human exists three-dimensionally (e.g., circumferentially) around the torso. That is, heart-caused signals may be detectable on the surface skin of the human at the chest, back, shoulders, under-arm areas, and the like.

Although biometric sensor infused garment embodiments of the present disclosure are described with respect to sensors arranged about the heart-active region 102 of a subject, it is recognized that the teaching herein is not so limited. Accordingly, such teaching may suitably be applied to the heart-active region and additionally, or alternatively, any one or more of a subject's head (e.g., frontal, parietal, occipital, or temporal region), neck, shoulder, back, upper arm, elbow, forearm, wrist, back-of-hand, palm, finger, thumb, hip, groin, thigh, calf, ankle, foot, and toe. Along these lines, the present disclosure describes devices, methods, and systems for collecting and processing biometric data such as heart-caused signals.

Furthermore, embodiments of the present disclosure are described with respect to human subjects, but the teaching presented herein may be understood more broadly to apply to any living subject. For example, biometric sensor infused garments having properties of the present disclosure may be arranged for non-human subjects such as livestock (e.g., bovine, swine, and any other type of livestock, wild animals, domesticated animals, carnivores, herbivores, omnivores, primates, marsupials, birds, rodents, reptiles, fish, and the like).

FIGS. 2A-2G are various embodiments of a biometric sensor infused garment 106A-106F. The garments 106A-106F of FIG. 2 are each a clothing article (i.e., a shirt), which may be worn by an athlete, but as discussed herein, other types of biometric sensor infused devices may alternatively be formed. The clothing article is arranged for wear about the subject's torso, and the clothing article includes a first opening 108 for substantially encircling the neck area of the user wearing the clothing article. A second opening 110 will substantially encircle the right extremity of the user (e.g., right arm, right shoulder), a third opening 112 will substantially encircle the left extremity of the user, and a fourth opening 114 will substantially encircle a lower portion of the torso of the subject (e.g., the lower stomach or waist area).

The garments 106A-106F of FIG. 2 each include a “band” integrated at the heart-active region 102 of the clothing article. To be clear, a garment may have a heart-active region, a band may have a heart-active region, and the body of any particular subject may have a heart-active region. With respect to a garment, a band, or any other structure, the heart-active region is an area of the structure where any suitable sensor may be positioned to detect heart-caused signals.

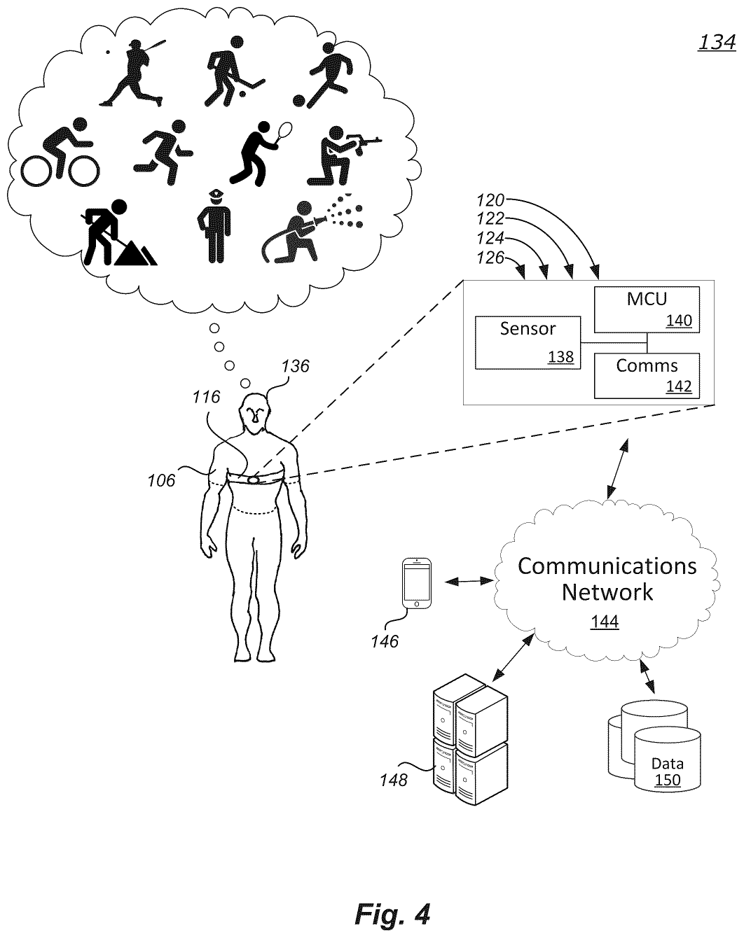

The band 116A-116F is arranged to contain the at least one biometric sensor 120 (FIG. 4) that is positioned in the heart-active region 102 where heart-caused signals are detectable. In many cases, the band 116A-116F is positioned for contact with the anterior part of the subject's anatomy (e.g., chest, front). In other cases, the band 116A-116F is positioned for contact with the posterior part of the subject's anatomy (e.g., back). In still other cases, the band 116A-116F may be arranged to collect heart-caused signals or any other biometric data from some other portion of the subject's body.

In FIG. 2A, the band 116A has a side-high configuration. In this configuration, right and left sides of the band 116A (i.e., areas of the band 116A generally in the respective armpit regions of the subject) make contact with the subject at a higher portion of the subject's torso than the center portion of the band 116A (i.e., area or areas of the band 116A generally in the central abdomen region of the subject).

In FIG. 2B, the band 116B has a center-high configuration. In this embodiment, a center portion of the band 116B contacts the subject at a higher portion of the subject's torso than right and left sides of the band 116B.

In the present embodiments, use of the relative terms, “higher,” “lower,” and the like are understood with reasonable certainty when each of the particular points or areas of interest are compared to a same point of reference, such as the floor, ground, waist area, or the like at a particular point or in a particular orientation. For example, in FIG. 2A, the distance between the sides of band 116A from a floor is greater than the distance between the center portion of band 116A from the floor when the subject is wearing the biometric sensor infused garment 106A. Conversely, in FIG. 2B, the distance between the sides of band 116B from the floor is less than the distance between the center portion of band 116A from the floor when the biometric sensor infused garment 106B is being worn. Similar analysis of terms will be understood by those of skill in the art.

In FIG. 2C, the band 116C has a linear configuration where the center portion of the band 116C contacts the subject at about the same height on the subject's torso as the right and left sides of the band 116C.

In FIG. 2D, the band 116D has a wide configuration, and in FIG. 2E, the band 116E has a narrow configuration. In the embodiments of FIG. 2, a band having a width of about three inches (3″) or more may be considered “wide;” a band having a width of about three inches (3″) or less may be considered “narrow;” and hence, a band having a width of about three inches (3″) may be considered narrow or wide. In other cases, such as when a band arranged to contain at least one biometric sensor is integrated or otherwise positioned on a human wrist or finger, a difference between “wide,” “narrow,” and other such terms of reference will generally be determined on a smaller dimension than three inches (3″). Along these lines, when a biometric sensor band is arranged for use with a cow, an elephant, a whale, or some other such subject, terms of referential size (e.g., “wide,” “narrow,” “big,” “small,” “close,” “far,” and the like) will generally be determined on a larger dimension than three inches (3″).

Although embodiments described herein and depicted in the figures show particular exemplary bands, other shapes and configurations are of course contemplated within the teaching of the present disclosure. Such bands, for example, may be symmetric or asymmetric, rectangular, triangular, circular, ovular, polygonal, circumferential, or any other suitable shape.

FIGS. 2F and 2G are, respectively, front and back embodiments of yet one more biometric-sensor infused garment 106F. The garment 106F includes a band 116F with a plurality of sub-structures. In at least some cases, the band 116F may itself be considered a “garment” as the term is used in the present disclosure. When the garment is worn by a subject, at least a portion of the band 116F is positioned at that heart-active region of the subject.

The band 116F comprises a superstructure 160, which may include any suitable number of layers as may be desirable in a particular garment. In at least one embodiment, a superstructure 160 has three layers. In other embodiments, a superstructure 160 has two layers, four layers, or some other number of layers. The superstructure 160 of FIG. 2F includes a first film 162 layer and a second film 164 layer. The first film layer 162 provides a substrate to which a second, smaller film layer 164 is added. The second film layer 164 forms at least one pocket (e.g., pouch, sleeve, bag, envelope, compartment, hollow, cavity, vessel, holder, repository, sheath, or the like) proximate the first film layer 162. The films 162, 164 may be a same one or different ones of a fabric, a plastic, or some other material. The films 162, 164 can be welded (e.g., heat welded, seam welded, glued, or welded in some other way), stitched or otherwise sewn, or joined in some other way.

In some cases, one or more of the films may be laser-cut to a particular shape and size and with a desirable set of features. In the embodiment of FIG. 2F, the first film 162 forms a generally rectangular substrate on which the second film 164 is positioned. The second film 164 is shaped having a wider central region and narrower right and left opposing regions. Other sizes, shapes, dimensions, and configuration are contemplated.

The at least one pocket of superstructure 160 is arranged to receive a biometric sensor 120. The biometric sensor 120 may be positioned inside the pocket via a slit, an aperture, an un-welded or un-stitched boundary, or some other force amplification receptacle access point 124. Within the pocket, at least one force amplification receptacle 122 is arranged to receive a respective one or more force amplification structures 126. In the embodiment of FIG. 2F, a single force amplification receptacle 122 may span to both of the narrower right and left opposing regions across the wider central region of the second film 162. In other cases, first and second force amplification receptacles 122 are formed, respectively, in the narrower right and left opposing regions.

The band 116F in the embodiment of FIGS. 2F, 2G is arranged to provide different levels of compression in different zones defined on the band 116F. In some cases, a first zonal compression region 166A (FIGS. 2F, 2G) is arranged as a sequence of apertures, thin areas, or some other structure. Sometimes, the pattern of apertures is symmetrical, and sometimes, the pattern of apertures is asymmetrical (e.g., random, patterned, positioned in accordance with a mathematical algorithm). As evident in FIG. 3G, a first zonal compression region 166A is adjacent a second zonal compression region 166B, which itself is adjacent a third zonal compression region 166C. More or fewer zonal compression regions are of course contemplated. To avoid unnecessarily cluttering the figure, only a single aperture in each of the first, second, and third zonal compression regions 166A, 166B, 166C are called out, but those of skill in the art will recognize that one or more of any such aperture are arranged to form the particular zonal compression region of interest.

Zonal compression regions can be formed in many ways. In the embodiment of FIG. 2G, apertures having a determined size are arranged in a close, symmetrical pattern to form the first zonal compression region 164A. Slightly larger, staggered apertures form the second zonal compression region 164B, and still larger apertures placed in an even less predictable pattern form the third zonal compression region 164C. By arranging the apertures 164A-164C in a manner that includes larger and more staggered apertures toward a front center, rear center, or front and rear center of the band 116F, compression lessens toward the front and back center of the garment 106F, and compression increases toward the right and left sides of the garment 106F. Other configurations are also contemplated.

FIGS. 3A and 3B are embodiments of a band structure 116G, 116H useful in one or more embodiments of a biometric infused garment. The band structures 116G, 116H are each arranged to contain at least one biometric sensor. The band structure 116G of FIG. 3A has a continuous configuration, and the band structure 116H of FIG. 3B has a segmented configuration. To avoid unnecessarily cluttering or otherwise obscuring the teaching of the present figures, certain structures common to one or more band structure embodiments are not expressly identified in each band structure.

A band structure 116 (FIG. 4) of the present disclosure is formed of any suitable material. Such band structure 116 may be arranged along the lines of any or all of band structures 116A-116H. In many cases, but not all cases, a band structure 116 is formed of a material suitable for a sporting activity, a dangerous activity, or any other such activities that require physical motion, strength, flexibility, and the like sufficient to cause detectable heart signals associated with the activity. In addition to sporting activities, or in the alternative to sporting activities, band structures as contemplated herein may be formed for activities such as, but not limited to, work, military service, medical service, first-responder service, or other activities that invoke any one or more of high physical stress, high mental stress, high emotional stress (i.e., activities associated with certain detectable heart signals).

Exemplary, but non-limiting, materials useful to create band structure 116 may comprise pure or blended textiles (e.g., cloths, fabrics, flannels, or the like) formed of natural or synthetic fibers. The textiles may include plant-based textiles (e.g., cotton, bamboo, flax, or the like), animal-based textiles (e.g., wool, silk, alpaca, and the like), synthetic-based textiles (e.g., polyester, nylon, spandex, and the like) or some other material. Individual portions (e.g., threads, yarns, strands, filaments, and the like) of a band structure 116 may include rubber or another material having suitable elastic properties. In at least some cases, the band structure material is formed as a separate or integral part of a compression shirt shaped to snugly fit the body of the subject.

The band 116G of FIG. 3A has an inside surface 118A and an outside surface 118B. The inside surface 118A is closer to the skin of the subject than the outside surface 118B. In some cases, the inside surface 118A is in direct contact with the body (e.g., the skin) of the subject when the band 116G is in use, and in other cases, one or more other substrates, layers, materials, and the like are between the inside surface 118A and the body of the subject.

In some cases, the outside surface 118B is exposed to the world and visible from the area proximate the subject, and in other cases, one or more other layers, materials, and the like are between the outside surface 118B and the outside world. In some cases, the band 116G, including its inside and outside surfaces 118A, 118B, are integrated with a garment as an integral part of said garment. In other cases, the band 116G is a separate and distinct structure that is temporarily or fixedly secured to the inside of a garment, to the outside of the garment, or between layers of the garment. In cases where the band is temporarily or fixedly integrated with a garment, the integration may be by an adhesive, a thread, a zipper, a clip, a button, a hook-and-loop mechanism, or any other suitable means.

The band 116G is arranged to cooperate with any suitable number of biometric sensors 120A, 120B. Two biometric sensors 120A, 120B are represented in the embodiment of FIG. 3A, but in other cases, a single biometric sensor, three biometric sensors, or some other number of biometric sensors is contemplated.

In the embodiment of FIG. 3A, heart-caused data may be detected by any or each of sensors 120A and 120B. In this and other such cases, analysis of the heart-caused data may require, or may be improved by, the association of particular data with the sensor that detected such data. For example, first signal information from data collected by a first biometric sensor 120A may be associated with a right-side of the subject, and second signal information from data collected by a second biometric sensor 120B may be associated with a left-side of the subject. Correspondingly, if band 116G is rotated 180 degrees such that biometric sensors 120A, 120B are situated on the posterior (i.e., back) of the subject, first signal information from data collected by a first biometric sensor 120A may be associated with a left-side of the subject, and second signal information from data collected by a second biometric sensor 120B may be associated with a right-side of the subject.

In some cases, biometric sensors, as taught in the present disclosure, are arranged as simple passive sensors, and in other cases, the biometric sensors are active electronic devices that may optionally comprise one or more sensing components, a power supply (e.g., a battery, a super-capacitor, or the like), a processor, memory, a transmitter or transceiver, and other circuitry. Accordingly, in some cases, biometric sensors are arranged to collect or generate data when stimulated by heart-caused signals of a subject (i.e., heart-data) for use by another electronic device, and in other cases, biometric sensors are local computing devices arranged to generate heart-data, store such heart-data, optionally process such heart-data, and communicate some or all of the raw or processed heart-data to another computing device.

One type of biometric sensor that may be used in embodiments of the present disclosure may include a multilayered flexible sensor having at least four layers. In such case, the first, outer layer is an insulating layer, the second layer is a flexible conductor layer, the third layer is a second insulating layer, and the fourth layer, which is arranged for placement against the skin of a subject, is manufactured as a comfortable, flexible, and moisture-permeable textile substrate. In such an arrangement, the conductor layer will include an electrode having a signaling surface proximate (e.g., a signaling surface that faces) the flexible substrate and makes contact with the skin of the subject via an aperture in the fourth textile substrate layer.

Considering the exemplary biometric sensor further, the flexible conductor layer (i.e., the second layer of the exemplary biometric sensor) may be insulated from liquid and other contaminants (e.g., perspiration that accumulates in the substrate during physical activity, moisture poured on the garment by the subject while drinking, dirt, dust, and the like) via the two insulating layers (i.e., the first and third layers of the exemplary biometric sensor). In such embodiment, the only portion of the second layer that is not sandwiched between the two insulating layers is the electrode portion.

Considering the exemplary biometric sensor further still, the substrate layer (i.e., the fourth layer of the exemplary biometric sensor) is preferably a textile or some other fiber, fibrous, or fiber-like material. The electrode may be formed of a metal, a conductive plastic, an elastomer, individual fibers, or a fiber material, such as a woven or knitted fabric. The second layer (i.e., the flexible conductor layer) may be formed of metal, a conductive plastic, a conductive fabric, a conductive rubber, a conductive elastomer, a conductive ink, a conductive polymer, a coating with a metal-particle content, a conductive fiber, or some other conductive means. The first and third layers (i.e., the insulating layers) may be formed of any material suitable for blocking moisture, particulates, electromagnetic signals, or anything that may interfere with the passage of heart-caused electrical signaling from the electrode to electronic circuitry that will collect and analyze the heart-caused electrical signals. Biometric sensors 120A, 120B are in many, but not all, cases configured to make direct contact with the body (e.g., the skin) of the subject. To this end, portions of biometric sensors 120A, 120B, or entire biometric sensors 120A, 120B, may be temporarily or permanently affixed to the band 116G. For example, biometric sensors 120A, 120B may be sewn into band 116G, stuck to band 116G with an adhesive (e.g., press-and-stick, iron-on, or the like), clipped to band 116G, attached to band 116G with hook-and-loop structures, or otherwise integrated with band 116G via some other means. In still other cases, biometric sensors 120A, 120B, or portions thereof, are temporarily or permanently integrated into a garment, and band 116G is arranged for placement “over” said sensors. As represented in FIG. 3A, biometric sensors 120A, 120B are shown with dashed lines to indicate that the sensors, when in use, are positioned between the band 116G and the subject on whom the sensors will collect data.

The band 116G in FIG. 3A includes a first force amplification receptacle 122A and a second force amplification receptacle 122B. In other embodiments, a band 116G will include a single force amplification receptacle, three force amplification receptacles, or some other number of force amplification receptacles. Additionally, each force amplification receptacle 122A, 122B includes a respective force amplification receptacle access point 124A, 124B. The amplification receptacles 122A, 122B are each arranged to receive a force amplification structure 126A via the respective force amplification receptacle access point 124A, 124B. In some cases, a force amplification receptacle 122A, 122B may not contain any force amplification structure 126A, 126B, and in other cases a force amplification receptacle 122A, 122B may contain one, two, or more force amplification structures 126A, 126B,

As taught in the present disclosure, the force amplification structures are arranged to promote (e.g., intensify, increase, stabilize, and the like) signal transmission between the body of the subject and the one or more biometric sensors. That is, as discussed herein, particularly (but not exclusively) in the heart-active region 102 (FIG. 1), heartbeat-caused electrical signals are detectable with certain sensors such as biometric sensors 120A, 120B. It has been learned by the inventors that weak contact, uncertain contact, incomplete contact, inconsistent contact, partially or completely non-existent contact, and other undesirable types of contact between the biometric sensors 120A, 120B and the body of the subject reduces the quality of biometric data collected by the sensors. Conversely, when directional area pressure is applied proximate the biometric sensors 120A, 120B, the quality of biometric data collected by the sensors is notably improved. Hence, while previous attempts have been made to tightly compress biometric sensors against the body of a subject via very strong elastic bands, adhesives, harnesses, and other contraptions, the present inventor has recognized that directionally increasing pressure (i.e., applying more pressure to the biometric sensors than the garment or band alone will apply to the biometric sensors) from substantially above a sensor and toward the body of the subject will lead to the collection of biometric sensor data having a desirable level of quality. Furthermore, the inventor has recognized that by applying more directional pressure to the area about the sensor relative to the pressure applied to other areas of the subject's body will lead to improved quality of biometric data collected by the sensors.

As considered in the present disclosure, “quality” of biometric data is a relative term. Biometric data of “high quality” is data that may be used to form an accurate determination of heart activity of a subject. Biometric data of “low quality” is data that in some cases is used to form an inaccurate or unreliable determination of heart activity of a subject, and in some cases, may not even be useful to form any opinion of heart activity. The accuracy and reliability of determined heart activity is generally based on whether or not the flow of electricity in a subject is detectable with accuracy and reliability. To this end, at least some of the factors that affect the accurate and reliable detection of electrical signals with biometric sensors include impedance between the electrode and the body of the subject, thermal noise, amplifier noise, interference, and baseline drift. Each of these factors may cause artifacts in the detected electrical signal data that reduce the data's quality, and accordingly, reducing the influence of any one or more of these factors will produce biometric data having improved quality. It has been further recognized by the inventor that in at least some cases, the effectiveness of a biometric sensor to produce high quality data may also be affected by the movement of the subject. For example, as the subject inhales, an expanded chest cavity volume may act to increase the quality of detected heart-active signals as contact between a sensor electrode and the subject's body is improved, and as the subject exhales, a contracted chest cavity volume may act to decrease the quality of detected heart-active signals as contact between a sensor electrode and the subject's body is diminished.

Considering one example, if a subject wears a snugly fitting t-shirt having at least one biometric sensor, the data collected by the sensor or sensors will have a first quality (i.e., a “t-shirt” quality); and if directional pressure as described herein is applied to the at least one biometric sensor, the data collected by the sensor or sensors will have a second quality (i.e., a “t-shirt-PLUS” quality), and the “t-shirt PLUS” quality data will be of higher quality than the “t-shirt” quality data.

Correspondingly, if the same subject wears a snugly fitting compression shirt having at least one biometric sensor, the data collected by the sensor or sensors will have a third quality (i.e., a “compression shirt” quality); and if directional pressure as described herein is applied to the at least one biometric sensor of the compression shirt, the data collected by the sensor or sensors will have a fourth quality (i.e., a “compression shirt PLUS” quality), and the “compression shirt PLUS” quality data will be of higher quality than the “compression shirt” quality data. Furthermore, in at least some cases, the data having the “t-shirt PLUS” quality may have a same or even higher quality that the data having the “compression shirt” quality. That is, in these cases, where directional pressure is applied in the t-shirt, the more comfortable, looser-fitting t-shirt may provide biometric sensor data of comparable or even higher quality than data produced using the tighter-fitting compression shirt.

Further still, the inventor has considered the case of a garment (e.g., a snug t-shirt, a fitted compression shirt, or some other garment) having at least one biometric-sensor integrated in a very high-compression band or harness. In this case, such band or harness applies a very high circumferential pressure around the subject's torso, and the data collected by the sensor or sensors will have a fifth quality (i.e., a “very high compression band” quality). But, different from previous garments, if directional pressure as described herein is applied to the sensor or sensors of the very high-compression band, the data collected by this sensor or these sensors (i.e., data having a sixth or “very high compression band PLUS” quality) will not be any higher than the data having the fifth quality. Thus, the inventor has learned that adding directional force to biometric sensors in a band that already applies a very high compression above a certain threshold to the subject will not yield data having higher quality than data collected in a corresponding band that does not apply directional pressure. That is, the force amplification systems described herein can improve the quality of collected biometric data, but the improvement has limits. Particularly, if a band or garment alone applies sufficient pressure to a biometric sensor, which threshold pressure is generally unique to each subject, then adding the force amplification structures described herein will not lead to biometric data having improved quality. What's more, and along these lines, the inventor has also learned that biometric data collected with a very high compression band may not be any better (i.e., higher quality) than data collected with a lower compression garment that deploys the force amplification systems of the present disclosure.

Hence, while the quality of collected biometric data may be improved by significantly increasing compression of an entire garment or an entire band wrapping the circumference of a subject, biometric data quality may also be improved by only increasing directional pressure in the area about the biometric sensor itself. And to this end, use of the force amplification structures described herein can improve the comfort of garments having biometric sensors without a corresponding sacrifice in the quality of collected data.

Considering the type of “directional pressure” described in the present disclosure, an effective increase in directional pressure is pressure that is applied substantially geometrically normal (e.g., between zero and forty-five degrees (0° to 45°) of normal) to the body of the subject where the sensor will collect data. Such directional pressure, which does not have to be perfectly normal the subject's body, improves both user comfort and the quality of data captured with a proximate biometric sensor. What's more, an increase in overall circumferential pressure caused by uncomfortable, very high compression, elastic bands, harnesses, buckles, and the like is not required.

As represented in the embodiment of FIG. 3A, the increased directional pressure of a biometric sensor 120A, 120B results from placement of the first force amplification structure 126A in the first force amplification receptacle 122A via first force amplification receptacle access point 124A, and, correspondingly, a second force amplification structure (not shown in FIG. 3A) in the second force amplification receptacle 122B via the force amplification receptacle access point 124B. The force amplification structures, once contained in their respective receptacles, cause increased directional pressure of the underlying respective biometric sensor 120A, 120B against the body of the subject.

Turning to FIG. 3B, one of skill in the art will recognize that several structures illustrated and described with respect to band 116G (FIG. 3A) are also found in band 116H, but such structures (e.g., portions of the band, inside and outside surfaces of the band, biometric sensors) are not identified so as to avoid unnecessarily cluttering the figure.

In FIG. 3B, band 116H includes a third force amplification receptacle 122C and a fourth force amplification receptacle 122D in dashed lines. The third and fourth force amplification receptacles 122C, 122D are different in form and placement from the first and second force amplification receptacles 122A, 122B of FIG. 3A. For example, while the first and second force amplification receptacles 122A, 122B are formed on the outside surface 118B of band 116G or at least accessible from the outside surface 118B of band 116G, the third and fourth force amplification receptacles 122C, 122D are formed within band 116H or on the inside surface 118B of band 116H. Along these lines, force amplification structures 126A-126G may be placed in use with a band 116 from either or both an inside surface 118A or an outside surface 118B.

Third force amplification receptacle 122C is arranged as an envelope-like structure, and fourth force amplification receptacle 122D is arranged as a set of containment strips (e.g., tabs, ribbons, slats, straps, or the like). Accordingly, a force amplification receptacle as contemplated herein may be arranged as a pocket, a pouch, a bag, an envelope, a compartment, a hollow, a cavity, a vessel, a holder, a repository, a sheath, or any other suitable receptacle formed from any suitable number of components. For example, the fourth force amplification receptacle 122D, which is illustrated in the embodiment of FIG. 3B as a set of four containment strips may alternatively be formed from one strip, two strips, three strips, or some other number of strips. As another example, the third force amplification receptacle 122C may form the envelope-like structure from a single piece of fabric, a pair of fabric pieces, or any suitable number of pieces of any suitable material.

Cooperative with the band embodiments of the present disclosure, several force amplification structure 126A-126G embodiments are also represented in FIG. 3B. Force amplification structure 126A has a first box-like structure with symmetric linear dimensions and a first length, a first width, and a first depth. Force amplification structure 126B has as second box-like structure with symmetric linear dimensions and a second length, a second width, and a second depth. Force amplification structure 126C has as third box-like structure with symmetric linear dimensions and a third length, a third width, and a third depth. The first depth is greater than the second and third depths, and the second depth is greater than the third depth. The first, second, and third lengths are a generally same length, and the first, second, and third widths are a generally same width. The length and width of any particular force amplification structure is generally determined to cooperate with a corresponding force amplification receptacle. The depth of any particular force amplification structure 126A-126C is generally determined based on a desired amount of directional pressure that will be applied to a respective biometric sensor. In some cases a single force amplification structure is used in a single force amplification receptacle. In other cases, two, three, or a different number of force amplification structures will be used in a single force amplification receptacle.

Additional non-limiting embodiments of force amplification structures are also represented in FIG. 3B. A fourth force amplification structure 126D is formed as a wedge. A fifth force amplification structure 126E is formed as an elliptical cylinder. A sixth force amplification structure 126F is formed as a cylinder. And a seventh force amplification structure 126G is formed as a wedge. Accordingly, force amplification structures as contemplated in the present disclosure, may have any suitable shape and any suitable dimensions.

Cooperative with the shape of a force amplification structure, the force amplification structures contemplated herein may be formed of any suitable material having any suitable properties. For example, in some cases, force amplification structures may be formed of plastic, nylon, foam, cardboard, metal, a composite, or any other suitable material. In some cases, a force amplification structure is rigid, and in other cases, a force amplification structure is flexible. In these and other cases, a force amplification structure may be curved or shaped to facilitate the directional force applied to an adjacent biometric sensor. For example, a force amplification structure may in some cases be contoured to follow the shape of the underlying biometric sensor or body of the subject of interest. In these and still other cases, a force amplification structure may include a guidance means (e.g., a well, an aperture, a shaped valley, a depression, a boss, a magnet, or some other like structure) to improve positioning of the force amplification structure. Generally speaking, the shape, dimensions, materials, and other properties of force amplification structures are selected to provide a desired amount of directional pressure that will be applied to a respective biometric sensor.

The force amplification structure in some embodiments may include other features. For example, in some cases, a force amplification structure is arranged as an energy storage device (e.g., a battery). In such cases, the battery may be used to power electronics of the related biometric sensor, consumer electronics, or some other electronic device. In some cases, other force amplification structures are arranged to provide cooling, weight (e.g., for training purposes), stability for anatomical structures (e.g., breasts), electronics (e.g., an audio device, a location circuit (e.g., global positioning system), a communication device, an audio feedback circuit, a haptic feedback circuit, or some other electronics), or yet some other structure.

As taught in the present disclosure, each force amplification structure may be arranged to cooperate with a given force amplification receptacle. To this end, some force amplification receptacles may be arranged with particular properties selected to improve the directional pressure properties associated with a respective biometric sensor. For example, in some cases, a force amplification receptacle may include a first surface proximal the body of the subject and a second surface distal the body of the subject. In these cases, the first and second surfaces of the force amplification receptacle at issue may be optionally formed from same materials or different materials. In at least one case, the first surface of the force amplification receptacle (i.e., the surface closest to the body of the subject) is formed from a soft, flexible material, and the second surface of the force amplification receptacle (i.e., the surface furthest from the body of the subject) is formed from a hard, rigid material. In this way, the force amplification structure is further biased to provide directional pressure of a respective biometric sensor.

In contrast to the continuous configuration band structure 116G of FIG. 3A, the band structure 116H of FIG. 3B has a segmented configuration. Band 116H is formed as a single piece of material having a generally rectangular shape and a cooperative linking mechanism 128A, 128B. In other embodiments, a band may be formed from two or more pieces of material and any suitable number of linking mechanisms.

The linking mechanism may include a single substructure, a pair of substructures, or a plurality of substructures, any number of which linking substructures may have same or different configurations. For example, the linking mechanism may include an adhesive, a hook, a catch, a loop, hook-and-loop structures, a clasp, a buckle, a hasp, a clip, a clamp, a fastener, or some other linking means.

Turning back to FIG. 3A, the continuous configuration band structure 116G is arranged as an unbroken, flexible belt-like structure. The band 116G, and additionally or alternatively any other band contemplated herein, may be arranged with a particular width 130, for example, between about one half inch (0.5″) and between about fifteen inches (15″). In at least one case, the width 130 of band 116G is about three to four inches (3″ to 4″). When a band as contemplated herein is configured for non-human use, the band may have any suitable width.

In the continuous configuration band structure 116G of FIG. 3A, the band 116G is may be arranged in a generally cylindrical form. While the band 116G is flexible and configured to conform to the body portion of interest of the subject when deployed, if the band 116G is otherwise understood in its cylindrical form (i.e., having a circular top-down cross section), then the band 116G may have a diameter between about five inches (5″) and about twenty inches (20″). When the band is configured for non-human use, the band may have any suitable diameter. Correspondingly, in the segmented configuration band structure 116H of FIG. 3B, the band 116H may be arranged in a generally rectangular form. In these cases, the band is deployed when it is wrapped around the selected body location of the subject and the linking mechanism 128A, 128B is engaged. In its rectangular orientation, however, the longest dimension of band 116H may in some cases have a length of between about eighteen inches (18″) and about sixty inches (60″). When the band is configured for non-human use, the band may have any suitable length.

FIG. 4 is a system 134 embodiment deploying a band structure 116 arranged to contain at least one biometric sensor 120, at least one force amplification receptacle 122, at least one force amplification receptacle access point 124, and at least one force amplification structure 126. The band structure 116 may be along the lines of any band structure described herein, having properties, for example, as any one or more of band structures 116A-116H (FIGS. 2, 3). The biometric sensor 120 may be along the lines of any biometric sensor described herein, such as the first and second biometric sensors 120A, 120B (FIG. 3). The force amplification receptacle 122, force amplification receptacle access point 124, and force amplification structure 126 may be along the lines of any such force amplification elements as taught herein, for example, force amplification receptacles 122A-122D (FIG. 3), force amplification receptacle access points 124A, 124B (FIG. 3), and force amplification structures 126A-126G (FIGS. 2, 3).

In FIG. 4, a subject 136 wears a biometric sensor infused garment 106. The garment 106 includes a band structure 116 having at least one optional force amplification receptacle 122, at least one optional force amplification receptacle access point 124, and at least one force amplification structure 126. In some cases, two or more force amplification structures 126 are contained in a single force amplification receptacle 122, and in other cases, a single force amplification structure 126 is contained in a single force amplification receptacle 122. At least one biometric sensor 120 is deployed, and such sensor may optionally be integrated in the band structure 116, the garment 106, or in some other way. In some cases, the band structure 116 is integrally formed with or as part of the garment 106, and in other cases, the band structure 116 is a separate and distinct structure that is temporarily or permanently deployed adjacent the garment 106.

The subject 136 in FIG. 4 may be equipped with the band structure 116 and other elements taught herein so that particular performance data related to the heart or other physiological data related to the subject may be collected. The subject 136 of FIG. 4 may be involved in an activity related to baseball, hockey, golf, soccer, kickball, biking, running, tennis, racquetball, archery, hunting, work (e.g., military, construction, law enforcement, firefighting, or any other work), or any other physically-, mentally-, or emotionally-stressful activity.

At certain times, at least one electronic sensor element 138 proximate the body of the subject 136 captures biometric (e.g., physiological, heart-signal, or the like) data associated with the subject 136. Electronic sensor element 138 may be arranged to capture any one or more of heart-signal data, temperature data, respiration data, oxygen saturation data, hydration data, motion data (e.g., accelerometer data, gyroscope data, or the like), or other biometric data associated with the subject 136.

Data collected by the one or more electronic sensor elements 138 are electronically processed. In some cases, the data is partially or fully processed by an optional processor such as microcontroller unit (MCU) 140. In such cases, raw data, processed data, or some combination of raw and processed data may be communicated to another computing device via communications circuitry 142.

Processing of data by the optional MCU 140 may include summing or otherwise accumulating data, averaging data, identifying data above or below a determined threshold (e.g., a selected heartrate, a selected temperature, a selected oxygen saturation level, and the like), combining data, generating a particular diagnosis about the subject based on the data (e.g., the subject's health is in danger, the subject is in a weight-loss zone, the subject is in a muscle-building zone, and the like), and generating other such conclusions. Other processing is of course contemplated.

In at least some cases, the MCU 140 may include peripheral circuitry of the type found in known microcontrollers. For example, the MCU 140 may include at least one processing core and memory. The memory, which may include volatile memory, non-volatile memory, or volatile and non-volatile memory, is arranged to store software instructions executable by the one or more processing cores. The memory may also store control information, calibration information, temporal data, datalogs, timing information, custom software applications, and any other data useful in operation of the biometric sensor device. In at least some cases, the peripheral circuitry may optionally also include inertial measurement units (e.g., single- or multi-axis accelerometers, single- or multi-axis gyroscopes, single- or multi-axis magnetometers), a power source (e.g., a battery, a super-capacitor, an induction-driven power supply, or the like), timers, input/output circuits, combinatorial logic, and any other suitable circuitry.

Data collected by the one or more electronic sensor elements 138, and additionally or alternatively data generated by MCU 140, may be communicated to an external computing device via communications circuitry 142. In some cases, the communications circuitry includes an electromechanical connector and a communications medium such as wire. In these and other cases, the communications circuitry 142 includes a wired or wireless transmitter, receiver, or transceiver operating in accordance with a proprietary or known protocol such as universal serial bus (USB), BLUETOOTH, or the like.

Data communicated from a band structure 116 may in some cases pass through a communications network 144. The communications network 144 may be a direct peer-to-peer communications network 144 such as a wire or cable (i.e., a single conduit or a multipath set of conduits). Alternatively, or additionally, the communications network 144 may be a shared communications network such as Ethernet or USB. In these and still other cases, the communications network 144 may be a wireless communications network operating under any suitable protocol (e.g., cellular, WiFi, BLUETOOTH, and the like).

Data communicated from a band structure 116 through a communications network 144 may be processed by a mobile computing device 146, a computing server 148, or both a mobile computing device 146 and a computing server 148. Data communicated from a band structure 116 through a communications network 144, and additionally or alternatively, data from one or both of a mobile computing device 146 and a computing server 148 may be stored in a data repository 150.

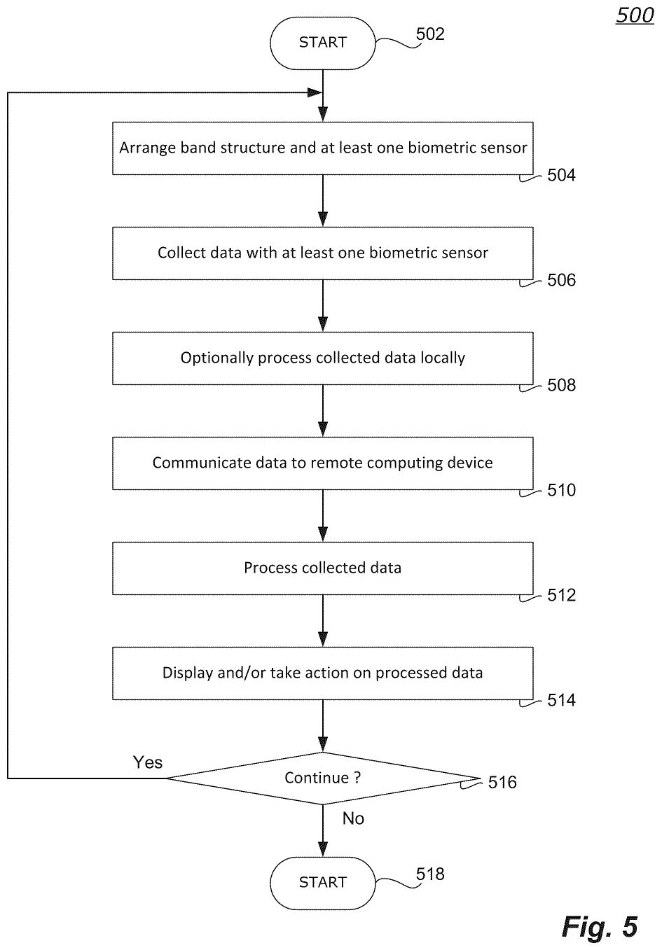

FIG. 5 is a data flow 500 diagram illustrating a non-limiting process that may be used by embodiments of a band structure system such as the system 134 illustrated in FIG. 4. Processing begins at 502.

At 504, a band structure is arranged for use on the body of a subject. The band structure includes at least one biometric sensor, and the operations of the at least one biometric sensor are enhanced by one or more force amplification structures having any suitable size, shape, material, and the like. In at least some cases, arrangement of the band structure may include initializing a system to collect data from the at least one biometric sensor, taking test measurement data, locating the band structure on the body of a subject, and adjusting the one or more force amplification structures to improve the quality, consistency, and reliability of collected biometric data. An MCU in some cases may cooperate with the biometric sensor to initialize, configure, or otherwise prepare the biometric sensor to collect, generate, or otherwise process high quality data. Processing falls to 506.

At 506, data is collected with at least one biometric sensor. The collected data may be any type of biometric data that is collectable with the selected one or more biometric sensors. For example, in cases where a biometric sensor is an electrocardiogram (ECG) sensor or some other capacitive electrode, the collected data may include representations of heart-caused signals (e.g., electrical activity of the heart captured over time).

In many cases, an ECG sensor is formed as a thin metal electrode with a thin oxide coating. This structure of ECG capacitive electrode is often selected because incomplete surface contact with the subject's body (e.g., skin) can still yield useful data, particularly if the surface contact is improved via use of the force amplification structures of the present disclosure. In some cases, the ECG sensor is formed of a flexible metal material. In these and other cases, signal conditioning and signal capturing electronic circuitry associated with the biometric sensor may also be formed of flexible material.

When an ECG biometric sensor is put into operation, changes in biological signals of the subject will electrostatically induce a charge in the sensor. As is known in the art, electrical signals of the subject's heart are stronger in closer proximity to the heart. Accordingly, placing sensors closer to the heart of the subject will provide stronger signals. Nevertheless, sensors placed farther from the heart may still provide useful data particularly if the force amplification structures of the present disclosure are deployed. Along these lines, in many cases, multiple biometric sensors are deployed (e.g., from two to twelve or more sensors), and at least one electrode of one sensor is driven to ground potential. In such configuration, the effects of noise may be reduced, which further improves operations when sensors are located farther from the subject's heart.

In some embodiments, one or more biometric sensors may capture temperature data. The temperature data may be associated with a subject, an environment, an object, or some other thing. For example, in some cases, temperature data representative of the body of the subject may be gathered. In these and other cases, ambient temperature in the area proximate the subject may be captured (e.g., to better understand athlete performance in various climates, geographical altitudes, times of day, and the like; to better understand a subject's performance in various ambient air scenarios; to better understand a subject's performance while wearing certain clothing that traps heat, releases heat, or the like; to better understand a fire fighter's performance in an active-fire area, inside the firefighters protective clothing, and the like; and to better understand temperature in other such circumstances).

In many cases, temperature sensors include metal-based (e.g., platinum, nickel, copper, and the like) conductive elements that change resistance under calibratable or otherwise known conditions. In these cases, a current may be applied to the element and surrounding circuitry, and a voltage may be measured across the active element of the sensor. In this way, the measured voltage may be used to generate one or more static, dynamic, or static and dynamic temperature values. Other temperature measurement sensors are also contemplated.

One or more biometric sensors may be configured to generate respiration data. Generally, a respiration sensor is arranged to produce data representative of a respiration signal, which is a relative measure of expansion of the subject's abdomen or thorax. Hence, in some cases, a biometric sensor as discussed herein may be arranged as a sensitive girth sensor that detects abdominal expansion and contraction (i.e., displacement) and generates respiration waveform and amplitude data representative of the subject's respiratory cycles, patterns, or cycles and patterns.

In at least one case, a biometric sensor may be arranged to collect data representative of a subject's peripheral oxygen saturation (i.e., how much of the hemoglobin in blood is carrying oxygen (SpO2)). Such sensors, which may be referred to as oximeters, pulse oximeters, or some other like term, typically include a light source and a light detector. The light source generates certain light (e.g., red light and infrared light) and directs such light through the biological material of the subject (e.g., a finger or some other part of a subject's anatomy). The light detector detects the amount of emitted light that passes through the portion of the subject's body that is associated with the sensor. As is known by those of skill in the art, in accordance with Beer's Law and Lambert's Law, the amount of light absorbed is proportional to the concentration of the light absorbing substance, and the amount of light absorbed is proportional to the distance light has to travel in the absorbing substance. Generally, oxygen-rich blood will absorb more of the transmitted light than oxygen-deprived blood. Such sensors, which collect light data over time are arranged to detect arterial blood flow, and this information may also be used to capture pulse data of the subject.

In some embodiments, one or more biometric sensors as contemplated herein may be arranged to generate hydration data associated with the subject. Such hydration data may be a rate of perspiration of the subject, a state of hydration of the subject, a core body temperature of the subject, a loss of electrolytes suffered by the subject, or some other hydration data.

In at least one case where a biometric sensor is arranged to generate hydration data, a sensor is arranged to collect and analyze bodily fluid (e.g., perspiration) of the subject, apply various excitation signals, and based on corresponding response signals, generate various impedance values. Such impedance values, which change over time as the subject performs certain stressful activities (e.g., physical activities, mental activities, emotional activities, and other activities) in particular environments, may be used to generate the hydration data.

Other sensor architectures arranged to collect different types of biometric data are also contemplated. For example, motion data associated with a subject 136 outfitted with a band structure 116 as described herein, may be collected with one or accelerometers, gyroscopes, gravity sensors, rotation sensors and the like. Such sensors, when used in the context of the present disclosure (i.e., sensors 138), produce a different type of physiological data associated with the subject 136. This motion data may be used alone or with other biometric data to inform on a particular health status of the subject 136.

After data is collected with at least one biometric sensor, processing advances to 508.

Processing at 508 is optional. Said processing, when included, is performed in cases where a band structure having at least one biometric sensor has circuitry arranged to conduct on-board data processing (e.g., an MCU and communications circuits (FIG. 4)). In these cases, such processing may include any one or more of collecting data over time, accumulating, averaging, storing, combining, discarding outlier data, and the like. Such processing may alternatively or additionally include one or both of receiving user input (e.g., button presses, vibration detection, and the like) and providing user output (e.g., audio, visual, tactile feedback and the like). Other processing functions are also contemplated. Processing advances to 510.

At 510, certain data from the band structure or a portion thereof is communicated to a remote computing device. Such communication may include transmission of the data over one or more conductive conduits (e.g., wires) in accordance with any known or proprietary protocol. Alternatively, or additionally, such communication may include transmission of the data wirelessly over one or more known or proprietary wireless communication protocols. Such communication may be performed in real time, periodically, or at some other desired rate, trigger, algorithm, random time, or the like.

The communication is optionally bidirectional. In such cases, the band structure or any circuitry therein may be arranged to receive control information, initialization information, or any other suitable data from a remote computing device. Such control information may include parameters, reset information, timing information, threshold information, or any other information selected for transmission to the band structure circuitry. Processing advances to 512.

At 512, the biometric data generated by the one or more biometric sensors and communicated from a band structure is processed in a known way. An analytic software application, for example, may be arranged to make calculations and inferences based on the received data in delayed time or in real time. Such calculations and inferences may be performed to better understand any number of physiological characteristics of the subject such as fitness level, internal training load, energy system development, heart rate variability (HVR), acute-to-chronic workload ratio (ACWR), state of recovery, and the like. The selected physiological characteristics may be monitored in real time or after certain determined activity, and in these cases, the selected physiological characteristics may be monitored over longer periods of time (e.g., days, weeks, months, over a season, and the like). Accordingly, the systems of the present disclosure, such as system 134 (FIG. 4) may be used for real-time monitoring, recovery monitoring, performance management, job safety evaluation, team competitions, and any other desired performance evaluations. Processing advances to 514.

At 514, action is taken based on the processing. In some cases, action is taken in real time. Real-time action may include, for example, triggering one or more alerts, making changes to controllable exercise equipment, making changes to controllable safety equipment, and performing other such actions. Exemplary alerts may optionally include triggering any one or more of visual alerts (e.g., illuminating a light source, flashing a light source, changing a video screen, or the like), audible alerts, or haptic (e.g., tactile) alerts. Such alert information may be communicated locally or to a remote device. Examples of controlling exercise equipment may include changing resistance on an exercise bike or stair-climber, changing speed on a treadmill, adjusting force required to move certain weight-lifting equipment, and the like. Examples of controlling safety equipment include, for example, adjusting an oxygen tank regulator, asserting a shut-down switch, and the like. Other actions are of course contemplated. Processing advances to 516.

At 516, the data flow 500 may continue or terminate. If the data flow 500 terminates, processing ends at 518. If the data flow 500 continues, processing advances back to 504. The decision whether to continue or terminate may be based on any suitable criteria. An exemplary list includes, but is not limited to, user interaction, detection of motion, determination of an absence of motion, power level, time, temperature, volume of data collected, quality of data collected, remote control, and the like.

In view of the embodiments illustrated and described herein, it is recognized by those of skill in the art that a band arranged to contain at least one biometric sensor may be formed as a band that is separate and distinct from a garment that is worn by a subject. In such cases, the band may be temporarily or permanently affixed to the garment. Alternatively, the band arranged to contain at least one biometric sensor may be a portion of the garment itself rather than a separate and distinct structure.

In further view of the embodiments of the present disclosure, it is also recognized by those of skill in the art that a band may be a fully circumferential structure arranged to fully encircle a torso, limb, or other portion of the subject's anatomy. In such configurations, the band may be permanently arranged as a fully circumferential structure (i.e., a continuous band structure), or arranged having one or more segments with two or more ends that are joined to create the fully circumferential structure. In other cases, the band may not be a circumferential structure at all. That is, the band may be a flat structure, rectangular structure, or a structure of any other shape that does not completely encircle the torso, head, neck, arm, leg, finger, snout, tail, or other portion of the subject of interest that is collecting biometric data.

As described herein, a band 116 may be formed of any suitable material, and the band may have any suitable dimensions, any suitable number of force amplification receptacles, and any suitable number of force amplification structures. Materials and other parameters of a band are selected to produce a desirable directional pressure on one or more biometric sensors.

The present inventor has developed several prototypes. Each prototype has yielded additional information and inspired particular changes (e.g., the addition of features, the removal of features, the changes to one or more dimensions, the determination that certain features are optional, and the like. In one exemplary case, the present inventor has developed a prototype garment which, when put in use with a particular subject, provided acceptable data.

This particular exemplary prototype, which is non-limiting, and which is described to inform those of skill in the art, is along the lines of the garment 106C of FIG. 2C. In such exemplary prototype, the band 116C has a linear configuration. Here, garment 106C is formed as long-sleeve athletic t-shirt generally arranged as a multi-use, performance-fit compression rash guard athletic garment, manufactured by LAFROI, and having a size labeled “Large” or “LG.” The exemplary t-shirt is made substantially of a blend of eighty percent (80%) synthetic polymer material (e.g., nylon) and twenty percent (20%) elastane material (e.g., DACRON, LYCRA, ORLON, or generically, spandex). The band 116C of the exemplary prototype is formed substantially from a polyester material infused with natural rubber latex strands, but any material having any suitable elastic properties could also have been selected. Generally, elasticity of the band 116C may be measured based on the amount of recoverable tensile strain under particular stress conditions. Here, the tensile strain is caused by deformation of the band 116C from its at-rest state (when garment 106C is not worn) to its stressed state (i.e., when garment 106C is donned by the subject). Where a first material, such as a first band, has a higher recoverable tensile strain under stress than a second material, such as a second band, the first material may be referred to as having a “stronger” elastic or stronger elastic properties than the second material. Generally, as the recoverable tensile strain of a material increases, the strength of the elastic of the material is understood to be increasing. Further analysis is omitted to avoid unnecessarily cluttering the present disclosure.

In this exemplary prototype, the band 116C is also formed as a segmented band, and there is no clasp; instead, the band 116C is sewed into the garment 106C. Band 116C is formed substantially of polyester with natural rubber latex having suitable elastomeric properties. The segmented band structure is about thirty-five and one-half inches (35.5″) long, three inches (3″) wide, and one thirty-second of an inch ( 1/32″) thick. The band 116C includes two biometric sensors, two force amplification receptacles, and two force amplification structures.

Though not illustrated in FIG. 2C, at least one embodiment is conceived having a draw string (e.g., a cord, a rope, a string, or the like, which may optionally include elastic properties) embedded in band 116C along with a cooperative cinching structure such as a cinch clip, a fastener, or some other cinching means that permits temporarily tightening band 116C. The optional draw string may extend fully or partially through the length of band 116C. In at least some cases, a subject may interactively tighten or loosen a draw string or other mechanism while a biometric sensor is capturing data and providing feedback on the quality of such data. In this way, a subject may arrange a garment for comfort while concurrently being assured that data collected by a biometric sensor will be useful.

Having now set forth certain embodiments, further clarification of certain terms used herein may be helpful to providing a more complete understanding of that which is considered inventive in the present disclosure.

In the embodiments of present disclosure, one or more particular structures, which may be textiles of one kind or another, include one or more biometric sensors and optionally include other electronic circuitry too. Various components and devices of the embodiments may be interchangeably described herein as “coupled,” “connected,” “attached,” and the like. It is recognized that once assembled, the system may include a band, a garment, and circuitry such as sensors, a power source, processing means, communications means, electromechanical coupling structures (e.g., connectors, clips, and the like). The materials and the junctions formed at the point where two or more structures meet in the present embodiments are connected and optionally sealed to a mechanically, medically, or otherwise industrially acceptable level.

FIG. 5 includes a data flow diagram illustrating a non-limiting process that may be used by embodiments of a band structure system such as the one illustrated in FIG. 4. In this regard, each described process may represent a module, segment, or portion of software code, which comprises one or more executable instructions for implementing the specified logical function(s). It should also be noted that in some implementations, the functions noted in the process may occur in a different order, may include additional functions, may occur concurrently, and/or may be omitted.

The figures in the present disclosure illustrate portions of one or more non-limiting computing device embodiments such as one or more components of system embodiment 134 (FIG. 4). The computing devices may include operative hardware found in conventional computing device apparatuses such as one or more processors, volatile and non-volatile memory, serial and parallel input/output (I/O) circuitry compliant with various standards and protocols, wired and/or wireless networking circuitry (e.g., a communications transceiver), one or more user interface (UI) modules, logic, and other electronic circuitry.

Processing devices, or “processors,” as described herein, include central processing units (CPU's), microcontrollers (MCU), digital signal processors (DSP), application specific integrated circuits (ASIC), peripheral interface controllers (PIC), state machines, and the like. Accordingly, a processor as described herein includes any device, system, or part thereof that controls at least one operation, and such a device may be implemented in hardware, firmware, or software, or some combination of at least two of the same. The functionality associated with any particular processor may be centralized or distributed, whether locally or remotely. Processors may interchangeably refer to any type of electronic control circuitry configured to execute programmed software instructions. The programmed instructions may be high-level software instructions, compiled software instructions, assembly-language software instructions, object code, binary code, micro-code, or the like. The programmed instructions may reside in internal or external memory or may be hard-coded as a state machine or set of control signals. According to methods and devices referenced herein, one or more embodiments describe software executable by the processor, which when executed, carries out one or more of the method acts.

The present disclosure discusses several embodiments that include or otherwise cooperate with one or more computing devices. It is recognized that these computing devices are arranged to perform one or more algorithms to implement various concepts taught herein. Each of said algorithms is understood to be a finite sequence of steps for solving a logical or mathematical problem or performing a task. Any or all of the algorithms taught in the present disclosure may be demonstrated by formulas, flow charts, data flow diagrams, narratives in the specification, and other such means as evident in the present disclosure. Along these lines, the structures to carry out the algorithms disclosed herein include at least one processing device executing at least one software instruction retrieved from at least one memory device. The structures may, as the case may be, further include suitable input circuits known to one of skill in the art (e.g., keyboards, buttons, memory devices, communication circuits, touch screen inputs, and any other integrated and peripheral circuit inputs (e.g., accelerometers, thermometers, light detection circuits and other such sensors)), suitable output circuits known to one of skill in the art (e.g., displays, light sources, audio devices, tactile devices, control signals, switches, relays, and the like), and any additional circuits or other structures taught in the present disclosure. To this end, every invocation of means or step plus function elements in any of the claims, if so desired, will be expressly recited.

As known by one skilled in the art, a computing device has one or more memories, and each memory comprises any combination of volatile and non-volatile computer-readable media for reading and writing. Volatile computer-readable media includes, for example, random access memory (RAM). Non-volatile computer-readable media includes, for example, read only memory (ROM), magnetic media such as a hard-disk, an optical disk, a flash memory device, a CD-ROM, and/or the like. In some cases, a particular memory is separated virtually or physically into separate areas, such as a first memory, a second memory, a third memory, etc. In these cases, it is understood that the different divisions of memory may be in different devices or embodied in a single memory. The memory in some cases is a non-transitory computer medium configured to store software instructions arranged to be executed by a processor. Some or all of the stored contents of a memory may include software instructions executable by a processing device to carry out one or more particular acts.

The computing devices illustrated herein may further include operative software found in a conventional computing device such as an operating system or task loop, software drivers to direct operations through 1/O circuitry, networking circuitry, and other peripheral component circuitry. In addition, the computing devices may include operative application software such as network software for communicating with other computing devices, database software for building and maintaining databases, and task management software where appropriate for distributing the communication and/or operational workload amongst various processors. In some cases, the computing device is a single hardware machine having at least some of the hardware and software listed herein, and in other cases, the computing device is a networked collection of hardware and software machines working together in a server farm to execute the functions of one or more embodiments described herein. Some aspects of the conventional hardware and software of the computing device are not shown in the figures for simplicity.

Amongst other things, the exemplary computing devices of the present disclosure (e.g., mobile computing device 146 and computing server 148 of FIG. 4) may be configured in any type of mobile or stationary computing device such as a remote cloud computer, a computing server, a smartphone, a tablet, a laptop computer, a wearable device (e.g., eyeglasses, jacket, shirt, pants, socks, shoes, other clothing, hat, helmet, other headwear, wristwatch, bracelet, pendant, other jewelry), vehicle-mounted device (e.g., train, plane, helicopter, unmanned aerial vehicle, unmanned underwater vehicle, unmanned land-based vehicle, automobile, motorcycle, bicycle, scooter, hover-board, other personal or commercial transportation device), industrial device (e.g., factory robotic device, home-use robotic device, retail robotic device, office-environment robotic device), or the like. Accordingly, the computing devices include other components and circuitry that is not illustrated, such as, for example, a display, a network interface, memory, one or more central processors, camera interfaces, audio interfaces, and other input/output interfaces. In some cases, the exemplary computing devices may also be configured in a different type of low-power device such as a mounted video camera, an Internet-of-Things (IoT) device, a multimedia device, a motion detection device, an intruder detection device, a security device, a crowd monitoring device, or some other device.

When so arranged as described herein, each computing device may be transformed from a generic and unspecific computing device to a combination device arranged comprising hardware and software configured for a specific and particular purpose such as to provide a determined technical solution. When so arranged as described herein, to the extent that any of the inventive concepts described herein are found by a body of competent adjudication to be subsumed in an abstract idea, the ordered combination of elements and limitations are expressly presented to provide a requisite inventive concept by transforming the abstract idea into a tangible and concrete practical application of that abstract idea.

The embodiments described herein use computerized technology to improve the technology of biometric sensor device data collection and analysis, but other techniques and tools remain available to collect data from subjects and assess the subject's performance. Therefore, the claimed subject matter does not foreclose the whole or even substantial biometric data collection and analysis technological area. The innovation described herein uses both new and known building blocks combined in new and useful ways along with other structures and limitations to create something more than has heretofore been conventionally known. The embodiments improve on computing systems which, when un-programmed or differently programmed, cannot perform or provide the specific system features claimed herein. The embodiments described in the present disclosure improve upon known biometric data collection and biometric data analysis processes and techniques. The computerized acts described in the embodiments herein are not purely conventional and are not well understood. Instead, the acts are new to the industry. Furthermore, the combination of acts as described in conjunction with the present embodiments provides new information, motivation, and business results that are not already present when the acts are considered separately. There is no prevailing, accepted definition for what constitutes an abstract idea. To the extent the concepts discussed in the present disclosure may be considered abstract, the claims present significantly more tangible, practical, and concrete applications of said allegedly abstract concepts. And said claims also improve previously known computer-based systems that perform biometric data collection and biometric data analysis operations.

Software may include a fully executable software program, a simple configuration data file, a link to additional directions, or any combination of known software types. When a computing device updates software, the update may be small or large. For example, in some cases, a computing device downloads a small configuration data file to as part of software, and in other cases, a computing device completely replaces most or all of the present software on itself or another computing device with a fresh version. In some cases, software, data, or software and data is encrypted, encoded, and/or otherwise compressed for reasons that include security, privacy, data transfer speed, data cost, or the like.

Database structures, if any are present in the biometric data collection and biometric data analysis systems described herein, may be formed in a single database or multiple databases. In some cases hardware or software storage repositories are shared amongst various functions of the particular system or systems to which they are associated. A database may be formed as part of a local system or local area network. Alternatively, or in addition, a database may be formed remotely, such as within a distributed “cloud” computing system, which would be accessible via a wide area network or some other network.

Input/output (I/O) circuitry and user interface (UI) modules include serial ports, parallel ports, universal serial bus (USB) ports, IEEE 802.11 transceivers and other transceivers compliant with protocols administered by one or more standard-setting bodies, displays, projectors, printers, keyboards, computer mice, microphones, micro-electro-mechanical (MEMS) devices such as accelerometers, and the like.

In at least one embodiment, devices such as the processor 140 of FIG. 4 may communicate with other devices via communication over a network 144. The communications network 144 may involve an Internet connection or some other type of local area network (LAN) or wide area network (WAN). Non-limiting examples of structures that enable or form parts of a network include, but are not limited to, an Ethernet, twisted pair Ethernet, digital subscriber loop (DSL) devices, wireless LAN, Wi-Fi, Worldwide Interoperability for Microwave Access (WiMax), or the like.

In the present disclosure, memory may be used in one configuration or another. The memory may be configured to store data. In the alternative or in addition, the memory may be a non-transitory computer readable medium (CRM). The CRM is configured to store computing instructions executable by a processor of at least some of the biometric sensors described in the present disclosure. The computing instructions may be stored individually or as groups of instructions in files. The files may include functions, services, libraries, and the like. The files may include one or more computer programs or may be part of a larger computer program. Alternatively or in addition, each file may include data or other computational support material useful to carry out the computing functions of a biometric data collection and biometric data analysis system.

Buttons, keypads, computer mice, memory cards, serial ports, bio-sensor readers, touch screens, and the like may individually or in cooperation be useful to a scientific practitioner operating the system 134. The devices may, for example, input control information into the system. Displays, printers, memory cards, LED indicators, temperature sensors, audio devices (e.g., speakers, piezo device, etc.), vibrators, and the like are all useful to present output information to the scientific practitioner operating the system 134. In some cases, the input and output devices are directly coupled to the sensor 138 and electronically coupled to a processor or other operative circuitry. In other cases, the input and output devices pass information via one or more communication ports (e.g., RS-232, RS-485, infrared, USB, etc.).

As described herein, for simplicity, a scientific practitioner, a user, a subject, or the like may in some cases be described in the context of the male gender. It is understood that such persons can be of any gender, and the terms “he,” “his,” and the like as used herein are to be interpreted broadly inclusive of all known gender definitions. As the context may require in this disclosure, except as the context may dictate otherwise, the singular shall mean the plural and vice versa; all pronouns shall mean and include the person, entity, firm or corporation to which they relate; and the masculine shall mean the feminine and vice versa.

The terms, “real-time” or “real time,” as used herein and in the claims that follow, are not intended to imply instantaneous processing, transmission, reception, or otherwise as the case may be. Instead, the terms, “real-time” and “real time” imply that the activity occurs over an acceptably short period of time (e.g., over a period of microseconds or milliseconds), and that the activity may be performed on an ongoing basis (e.g., collecting electrical signal data from a body of a subject and processing, communicating, or processing and communicating such data). An example of an activity that is not real-time is one that occurs over an extended period of time (e.g., hours or days) or that occurs based on intervention, direction, or other activity of a scientific practitioner, a user, a subject, or some other party.

In the absence of any specific clarification related to its express use in a particular context, where the terms “substantial” or “about” in any grammatical form are used as modifiers in the present disclosure and any appended claims (e.g., to modify a structure, a dimension, a measurement, or some other characteristic), it is understood that the characteristic may vary by up to 30 percent or by some other value if such other value is expressly stated. For example, a band structure may be described as having a width of about three inches (3 in.). In these cases, a width of exactly three inches (3 in.) clearly meets the description. Different from the exact precision of the dimension, “three inches (3 in.),” the use of “about” to modify the characteristic permits a variance of the “three inches (3 in.)” characteristic by up to 30 percent or by some other value that is expressly stated. Accordingly, in this example, a width of “about three inches (3 in.)” includes band structures having a width between two and nine-tenths inches and three and nine-tenths inches (2.1 in. and 3.9 in., respectively). A band structure having a width that is less than two inches (2.0 in.) is not a width of “about three inches (3 in.), and a band structure having a width that is more than four inches (4.0 in.) is not a width of “about three inches (3 in.) either. As another example, a band structure having a particular linear dimension of “between about three (3) inches and five (5) inches” includes such structures in which the linear dimension varies by up to 30 percent, Accordingly, the particular linear dimension of the band structure may be between one point five (1.5) inches and six point five (6.5) inches.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, the technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

In the present disclosure, when an element (e.g., component, circuit, device, apparatus, structure, layer, material, or the like) is referred to as being “on,” “coupled to,” or “connected to” another element, the elements can be directly on, directly coupled to, or directly connected to each other, or intervening elements may be present. In contrast, when an element is referred to as being “directly on,” “directly coupled to,” or “directly connected to” another element, there are no intervening elements present.

The terms “include” and “comprise” as well as derivatives and variations thereof, in all of their syntactic contexts, are to be construed without limitation in an open, inclusive sense, (e.g., “including, but not limited to”). The term “or,” is inclusive, meaning and/or. The phrases “associated with” and “associated therewith,” as well as derivatives thereof, can be understood as meaning to include, be included within, interconnect with, contain, be contained within, connect to or with, couple to or with, be communicable with, cooperate with, interleave, juxtapose, be proximate to, be bound to or with, have, have a property of, or the like.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In the present disclosure, the terms first, second, etc., may be used to describe various elements, however, these elements are not be limited by these terms unless the context clearly requires such limitation. These terms are only used to distinguish one element from another. For example, a first machine could be termed a second machine, and, similarly, a second machine could be termed a first machine, without departing from the scope of the inventive concept.

The singular forms “a,” “an,” and “the” in the present disclosure include plural referents unless the content and context clearly dictates otherwise. The conjunctive terms, “and” and “or” are generally employed in the broadest sense to include “and/or” unless the content and context clearly dictates inclusivity or exclusivity as the case may be. The composition of “and” and “or” when recited herein as “and/or” encompasses an embodiment that includes all of the elements associated thereto and at least one more alternative embodiment that includes fewer than all of the elements associated thereto.

In the present disclosure, conjunctive lists make use of a comma, which may be known as an Oxford comma, a Harvard comma, a serial comma, or another like term. Such lists are intended to connect words, clauses or sentences such that the thing following the comma is also included in the list.

The headings and Abstract of the Disclosure provided herein are for convenience only and do not interpret the scope or meaning of the embodiments.

The biometric wearable device, method, and system embodiments described in the present disclosure provide several technical effects and advances to the field of heart-caused signal monitoring. Technical effects and benefits include the ability to improve the reliability, quality, or reliability and quality of collected information representative of the heart-caused signals of a particular subject. These improvements may be used to improve conditioning, endurance, strength, or other characteristics of a subject wearing embodiments of the devices described in the present disclosure. The subject may be an athlete, a medical patient, a front-line worker in a particularly stressful occupation (e.g., police officer, soldier, fire fighter, teacher, corporate executive, social worker, healthcare worker, pilot, engineer, and the like), a performer, person in the process of sleep, a patient undergoing a medical event or procedure, a non-human animal, or any other subject. The biometric wearable device may be arranged as a shirt, jacket, vest, suspender or pair of suspenders, pant, sock, hat, jewelry, eyeglasses, pad, sporting equipment, gear, or any other type of garment as that term is used in this present disclosure.

The present disclosure sets forth details of various structural embodiments that may be arranged to carry out the teaching of the present disclosure. By taking advantage of the force amplification receptacles, force amplification structures, flexible circuitry, mechanical structures, computing architecture, and communications means described herein, a number of exemplary devices and systems are now disclosed.

Example A-1 is a biometric wearable device comprises a garment arranged for wear about a torso of a subject, the garment including: a first opening for substantially encircling a neck area of the subject that is wearing the garment; a second opening for substantially encircling a right extremity of the subject; a third opening for substantially encircling a left extremity of the subject; a fourth opening for substantially encircling a lower portion of the torso of the subject; and a heart-active region which, when the subject is wearing the garment, is proximate an area of the torso of the subject where heart-caused signals are detectable. In such embodiment, the biometric wearable device further comprises a biometric sensor arranged to detect the heart-caused signals of the subject when the subject is wearing the garment; a band positioned at the heart-active region of the garment and arranged to contain the biometric sensor; and at least one force amplification structure positionable in a force amplification receptacle of the band, wherein the at least one force amplification structure is arranged to provide increased directional pressure to the biometric sensor.

Example A-2 may include the subject matter of Example A-1, and alternatively or additionally any other example herein, wherein the band is arranged to position an active portion of the at least one biometric sensor in direct contact with the torso of the subject.

Example A-3 may include the subject matter of any of Examples A-1 to A-2, and alternatively or additionally any other example herein, wherein the biometric sensor includes at least two electrodes.

Example A-4 may include the subject matter of any of Examples A-1 to A-3, and alternatively or additionally any other example herein, wherein the band is at least ten inches (10 in.) long.

Example A-5 may include the subject matter of any of Examples A-1 to A-5, and alternatively or additionally any other example herein, wherein the band is less than sixty inches (60 in.) long.

Example A-6 may include the subject matter of any of Examples A-1 to A-5, and alternatively or additionally any other example herein, wherein the band is a continuous band having at diameter between about eight to fifteen inches (8 in. to 15 in.).

Example A-7 may include the subject matter of any of Examples A-1 to A-6, and alternatively or additionally any other example herein, wherein the band is between about one to four inches (1 in. to 4 in.) wide.

Example A-8 may include the subject matter of any of Examples A-1 to A-7, and alternatively or additionally any other example herein, wherein the band is between about one to five hundred mils (0.001 in. to 0.5 in.) thick.

Example A-9 may include the subject matter of any of Examples A-1 to A-8, and alternatively or additionally any other example herein, wherein the band is formed from a material having elastic properties.

Example A-10 may include the subject matter of any of Examples A-1 to A-9, and alternatively or additionally any other example herein, wherein the band is formed from a first material having stronger elastic properties than a second material used to form the garment.

Example A-11 may include the subject matter of any of Examples A-1 to A-10, and alternatively or additionally any other example herein, wherein the band exposes at least one electromechanical structure arranged to pass signal information associated with the at least one biometric sensor.

Example A-12 may include the subject matter of any of Examples A-1 to A-11, and alternatively or additionally any other example herein, wherein the at least one electromechanical structure is a wire or an electrical connector.

Example A-13 may include the subject matter of any of Examples A-1 to A-12, and alternatively or additionally any other example herein, wherein the garment is a shirt.

Example A-14 may include the subject matter of any of Examples A-1 to A-13, and alternatively or additionally any other example herein, wherein the subject is a human being.

Example A-15 may include the subject matter of any of Examples A-1 to A-14, and alternatively or additionally any other example herein, wherein the subject is a non-human mammal.

Example A-16 may include the subject matter of any of Examples A-1 to A-15, and alternatively or additionally any other example herein, wherein the biometric sensor is one of a plurality of biometric sensors integrated in the band.

Example A-17 may include the subject matter of any of Examples A-1 to A-16, and alternatively or additionally any other example herein, wherein garments and biometric data collection devices may optionally include any one or more of shirts, jackets, vests, suspenders or pairs of suspenders, pants, socks, hats, jewelry, glasses, pads, sporting equipment, gear, sensors, inspection points, computing devices, circuitry, wired and wireless communications protocols, wired and wireless transceivers, radios, communications ports, geolocation, and other such electronic, electrical, electromechanical, mechanical, and data collection means.

Example A-18 may include the subject matter of any of Examples A-1 to A-17, and alternatively or additionally any other example herein, wherein the devices, systems, and methods to collect biometric data of the subject are improved when the sensors are in firm, stable contact with the subject's body, and the garment (i.e., the structure or structures containing or otherwise positioning and/or securing the sensor or sensors) is comfortable and easy to use.

Example A-19 may include the subject matter of any of Examples A-1 to A-18, and alternatively or additionally any other example herein, wherein a garment reliably and comfortably places one or more biometric sensors in firm, stable contact with the subject's body during certain biometric data collection processes.

Example A-20 may include the subject matter of any of Examples A-1 to A-19, and alternatively or additionally any other example herein, wherein any one or more of the biometric sensors are arranged to detect at least one signal defined as an electrical impulse, a voltage, a current, a wave, an excitation, a stimulation, or the like.

Example A-21 may include the subject matter of any of Examples A-1 to A-20, and alternatively or additionally any other example herein, wherein the biometric wearable device, which may comprise a garment, such as a clothing article or some other garment, is arranged for placement on any one or more of a subject's head (e.g., frontal, parietal, occipital, or temporal region), neck, shoulder, back, upper arm, forearm, wrist, back-of-hand, palm, hip, groin, thigh, calf, ankle, foot, or any other part of the subject's body.

Example A-22 may include the subject matter of any of Examples A-1 to A-21, and alternatively or additionally any other example herein, wherein the biometric wearable device, which includes one or more biometric sensors, is arranged as a hat, a headband, another type of headwear, a scarf, a shirt, a vest, a suspender or pair of suspenders, a jacket, a jersey, a pant or pair of pants, a legging or pair of leggings, a short or pair of shorts, an undergarment, a sock or pair of socks, a shoe or pair of shoes or other footwear, a band (e.g., an armband, a leg band, or any other band-like structure), a glove or pair of gloves, a mitten or pair of mittens, a wristwatch or some other wrist-worn structure, jewelry, protective padding, and or some other activity-related equipment, which may include, but is not limited to: body armor, breathing apparatus such as a tank, safety or other protective gear, control equipment such as a steering wheel or joystick, a comfort structure such as a chair, a beds, or the like.

Example A-23 may include the subject matter of any of Examples A-1 to A-22, and alternatively or additionally any other example herein, wherein the subject is a human or a non-human subject.

Example A-24 may include the subject matter of any of Examples A-1 to A-23, and alternatively or additionally any other example herein, wherein the subject is a non-human subject drawn from the non-limiting list of livestock (e.g., bovine, swine, and any other type of livestock), wild animals, domesticated animals, carnivores, herbivores, omnivores, primates, marsupials, birds, rodents, reptiles, fish, and the like.

Example A-25 may include the subject matter of any of Examples A-1 to A-24, and alternatively or additionally any other example herein, wherein the subject is any one of an athlete, a soldier, any type of a front-line emergency worker (e.g., a law enforcement officer, a prison guard, a doctor, a nurse, a healthcare provider, a fire fighter, and the like), or any other type of worker that may act in a circumstance that an ordinary person may find physically, mentally, or emotionally stressful, including, but not limited to, a social worker, a delivery person, a messenger, a public speaker, a pilot, a truck driver, a captain of a vessel, a performer, person in the process of sleep, a patient undergoing a medical event or procedure, a non-human animal, or any other subject.

Example A-26 may include the subject matter of any of Examples A-1 to A-25, and alternatively or additionally any other example herein, wherein the band has a high-side configuration, a center-high configuration, or a linear configuration.

Example A-27 may include the subject matter of any of Examples A-1 to A-26, and alternatively or additionally any other example herein, wherein the band has a width of about three inches (3″).

Example A-28 may include the subject matter of any of Examples A-1 to A-27, and alternatively or additionally any other example herein, wherein the band is generally symmetric.

Example A-29 may include the subject matter of any of Examples A-1 to A-28, and alternatively or additionally any other example herein, wherein the band is generally asymmetric.

Example A-30 may include the subject matter of any of Examples A-1 to A-29, and alternatively or additionally any other example herein, wherein the band is has at least one portion that is generally symmetric and at least one portion that is generally asymmetric.

Example A-31 may include the subject matter of any of Examples A-1 to A-30, and alternatively or additionally any other example herein, wherein the band is has at least one portion that is generally rectangular, triangular, circular, ovular, polygonal, circumferential, or of some other suitable shape.

Example A-32 may include the subject matter of any of Examples A-1 to A-31, and alternatively or additionally any other example herein, wherein the band structure has a continuous configuration

Example A-33 may include the subject matter of any of Examples A-1 to A-32, and alternatively or additionally any other example herein, wherein the band structure has a segmented configuration.

Example A-34 may include the subject matter of any of Examples A-1 to A-33, and alternatively or additionally any other example herein, wherein the band structure is formed of a material suitable for a sporting activity or other such activities that require physical motion, strength, flexibility, and the like.

Example A-35 may include the subject matter of any of Examples A-1 to A34, and alternatively or additionally any other example herein, wherein the band structure is formed of a material suitable for activities such as, but not limited to, work, military service, medical service, or other activities that invoke any one or more of high physical stress, high mental stress, and high emotional stress.

Example A-36 may include the subject matter of any of Examples A-1 to A-35, and alternatively or additionally any other example herein, wherein the band structure is formed, entirely or partially, from any one or more of pure or blended textiles (e.g., cloths, fabrics, flannels, or the like) comprising natural or synthetic fibers, wherein such textiles may include any one or more of plant-based textiles (e.g., cotton, bamboo, flax, or the like), animal-based textiles (e.g., wool, silk, alpaca, and the like), and synthetic-based textiles (e.g., polyester, nylon, spandex, and the like).

Example A-37 may include the subject matter of any of Examples A-1 to A-36, and alternatively or additionally any other example herein, wherein the band structure is formed, entirely or partially, from any one or more of threads, yarns, strands, filaments, and the like, which themselves may comprise any one or more of rubber or another material having suitable elastic properties.

Example A-38 may include the subject matter of any of Examples A-1 to A-37, and alternatively or additionally any other example herein, wherein the band structure has an inside surface and an outside surface, and wherein the inside surface is closer to the skin of the subject than the outside surface. In some cases, the inside surface 118A is in direct contact with the body (e.g., the skin) of the subject when the band 116F is in use, and in other cases, one or more other substrates, layers, materials, and the like are between the inside surface 118A and the body of the subject.

Example A-39 may include the subject matter of any of Examples A-1 to A-38, and alternatively or additionally any other example herein, wherein the band structure has an inside surface and an outside surface, and wherein the inside surface is in direct contact with the body (e.g., the skin) of the subject when the band structure is in use.

Example A-40 may include the subject matter of any of Examples A-1 to A-39, and alternatively or additionally any other example herein, wherein the band structure has an inside surface and an outside surface, and wherein one or more other substrates, layers, materials, and the like are between the inside surface and the body of the subject.

Example A-41 may include the subject matter of any of Examples A-1 to A-40, and alternatively or additionally any other example herein, wherein the band structure has an inside surface and an outside surface, and wherein the outside surface is exposed to the world and visible from the area proximate the subject.

Example A-42 may include the subject matter of any of Examples A-1 to A-41, and alternatively or additionally any other example herein, wherein the band structure has an inside surface and an outside surface, and wherein one or more other layers, materials, and the like are between the outside surface and the outside world.

Example A-43 may include the subject matter of any of Examples A-1 to A-42, and alternatively or additionally any other example herein, wherein the band structure has an inside surface and an outside surface, and wherein the inside and outside surfaces are integrated with a garment as an integral part of said garment.

Example A-44 may include the subject matter of any of Examples A-1 to A-43, and alternatively or additionally any other example herein, wherein the band structure has an inside surface and an outside surface, and wherein the band structure is a separate and distinct structure that is temporarily or fixedly secured to the inside of a garment, to the outside of the garment, or between layers of the garment.

Example A-45 may include the subject matter of any of Examples A-1 to A-44, and alternatively or additionally any other example herein, wherein the biometric wearable device includes any suitable number of biometric sensors, which may include one biometric sensor, two biometric sensors, three biometric sensors, or some other number of biometric sensors.

Example A-46 may include the subject matter of any of Examples A-1 to A-45, and alternatively or additionally any other example herein, wherein any one or more of the biometric sensors has any suitable size, shape, and material composition.

Example A-47 may include the subject matter of any of Examples A-1 to A-46, and alternatively or additionally any other example herein, wherein any one or more of the biometric sensors has a cross-section or outline that is generally square, rectangular, circular, elliptical, ovular, polygonal, or some other geometric shape.

Example A-48 may include the subject matter of any of Examples A-1 to A-47, and alternatively or additionally any other example herein, wherein any one or more of the biometric sensors is formed of any one or more of plastic, metal, fabric, adhesive, and the like.

Example A-49 may include the subject matter of any of Examples A-1 to A-48, and alternatively or additionally any other example herein, wherein any one or more of the biometric sensors is arranged as a simple passive sensor.

Example A-50 may include the subject matter of any of Examples A-1 to A-49, and alternatively or additionally any other example herein, wherein any one or more of the biometric sensors is arranged as an active electronic device that may optionally comprise one or more sensing components.

Example A-51 may include the subject matter of any of Examples A-1 to A-50, and alternatively or additionally any other example herein, wherein any one or more of the biometric sensors includes one or more sensing components, and one or more of a power supply (e.g., a battery, a super-capacitor, or the like), a processor, memory, a transmitter or transceiver, and other such circuitry.

Example A-52 may include the subject matter of any of Examples A-1 to A-51, and alternatively or additionally any other example herein, wherein any one or more of the biometric sensors is arranged to collect or generate data when stimulated by heart-caused signals of a subject (i.e., heart-data) for use by another electronic device.

Example A-53 may include the subject matter of any of Examples A-1 to A-52, and alternatively or additionally any other example herein, wherein any one or more of the biometric sensors are arranged as local computing devices arranged to generate raw heart-data, store such raw heart-data, optionally process such raw heart-data into processed heart-data, and communicate some or all of the raw or processed heart-data to another computing device.

Example A-54 may include the subject matter of any of Examples A-1 to A-53, and alternatively or additionally any other example herein, wherein any one or more of the biometric sensors are configured to make direct contact with the body (e.g., the skin) of the subject.

Example A-55 may include the subject matter of any of Examples A-1 to A-54, and alternatively or additionally any other example herein, wherein any one or more of the biometric sensors are temporarily or permanently affixed to the band structure.

Example A-56 may include the subject matter of any of Examples A-1 to A-55, and alternatively or additionally any other example herein, wherein any one or more of the biometric sensors are sewn into the band structure, stuck to the band structure with an adhesive, clipped to the band structure, attached to the band structure with hook-and-loop structures, or otherwise integrated with the band structure via some other means.

Example A-57 may include the subject matter of any of Examples A-1 to A-56, and alternatively or additionally any other example herein, wherein the band structure any one or more of the biometric sensors, or portions thereof, are temporarily or permanently integrated into the garment (e.g., a clothing article), and the band structure is arranged for placement “over” said any one or more biometric sensors.

Example A-58 may include the subject matter of any of Examples A-1 to A-57, and alternatively or additionally any other example herein, wherein the garment (e.g., a biometric wearable device) includes one force amplification receptacle, two force amplification receptacles, three force amplification receptacles, or some other number of force amplification receptacles.

Example A-59 may include the subject matter of any of Examples A-1 to A-58, and alternatively or additionally any other example herein, wherein the garment (e.g., a biometric wearable device) includes any suitable number of force amplification receptacles, and wherein any one or more of the force amplification receptacles is arranged to receive one force amplification structure, two force amplification structures, three force amplification structures, or any suitable number of force amplification structures.

Example A-60 may include the subject matter of any of Examples A-1 to A-59, and alternatively or additionally any other example herein, wherein one or more force amplification structures cause a reduction in at least one of impedance, thermal noise, amplifier noise, interference, and baseline drift, which otherwise negatively affect the integrity of electrical signal data collected by at least one biometric sensor.

Example A-61 may include the subject matter of any of Examples A-1 to A-60, and alternatively or additionally any other example herein, wherein biometric sensor data collectable by a first biometric sensor has a first level of quality, and biometric sensor data collectable by the first biometric sensor when the first biometric sensor also has at least one adjacent force amplification structure applying direction pressure thereon has a second level of quality, the second level of quality being higher than the first level of quality.

Example A-62 may include the subject matter of any of Examples A-1 to A-61, and alternatively or additionally any other example herein, wherein the garment (e.g., clothing article) having the at least one a force amplification structure is generally more comfortable to the subject than a similar garment having no force amplification structures and a higher overall compression.

Example A-63 may include the subject matter of any of Examples A-1 to A-62, and alternatively or additionally any other example herein, wherein each force amplification structure is arranged to increase directional pressure on a corresponding biometric sensor, said directional pressure being pressure applied substantially geometrically normal (e.g., between zero and forty-five degrees (0⁰ to 45°) of normal) to the body of the subject.

Example A-64 may include the subject matter of any of Examples A-1 to A-63, and alternatively or additionally any other example herein, wherein one or more force amplification receptacles are formed on an outside surface of the band, an inside surface of the band, or both outside and inside surfaces of the band.

Example A-65 may include the subject matter of any of Examples A-1 to A-64, and alternatively or additionally any other example herein, wherein at least one amplification receptacle is arranged as an envelope-like structure, a set of containment strips (e.g., tabs, ribbons, slats, straps, or the like), a pocket, a pouch, a bag, an envelope, a compartment, a hollow, a cavity, a vessel, a holder, a repository, a sheath, or any other suitable receptacle formed from any suitable number of components.

Example A-66 may include the subject matter of any of Examples A-1 to A-65, and alternatively or additionally any other example herein, wherein at least one amplification receptacle is formed from one strip, two strips, three strips, or some other number of strips. As another example, the third force amplification receptacle may form the envelope-like structure from a single piece of fabric, a pair of fabric pieces, or any suitable number of pieces of any suitable material

Example A-67 may include the subject matter of any of Examples A-1 to A-66, and alternatively or additionally any other example herein, wherein at least one amplification receptacle is formed from one strip, two strips, three strips, or some other number of strips.

Example A-68 may include the subject matter of any of Examples A-1 to A-67, and alternatively or additionally any other example herein, wherein at least one amplification receptacle is formed from a single piece of fabric, a pair of fabric pieces, or any suitable number of pieces of any suitable material.

Example A-69 may include the subject matter of any of Examples A-1 to A-68, and alternatively or additionally any other example herein, wherein at least one force amplification structure has a first box-like structure with linear dimensions and a first length, a first width, and a first depth.

Example A-70 may include the subject matter of any of Examples A-1 to A-69, and alternatively or additionally any other example herein, wherein at least one force amplification structure is selected based on a desired amount of directional pressure that will be applied to a respective biometric sensor.

Example A-71 may include the subject matter of any of Examples A-1 to A-70, and alternatively or additionally any other example herein, wherein at least one force amplification structure is formed as a wedge, an elliptical cylinder, a cylinder, or another suitable shape having any suitable dimensions.

Example A-72 may include the subject matter of any of Examples A-1 to A-71, and alternatively or additionally any other example herein, wherein at least one force amplification structure is formed of any suitable material having any suitable properties.

Example A-73 may include the subject matter of any of Examples A-1 to A-72, and alternatively or additionally any other example herein, wherein at least one force amplification structure is formed of at least one of plastic, nylon, foam, cardboard, metal, a composite, or another suitable material.

Example A-74 may include the subject matter of any of Examples A-1 to A-73, and alternatively or additionally any other example herein, wherein at least one force amplification structure is rigid.

Example A-75 may include the subject matter of any of Examples A-1 to A-74, and alternatively or additionally any other example herein, wherein at least one force amplification structure is flexible.

Example A-76 may include the subject matter of any of Examples A-1 to A-75, and alternatively or additionally any other example herein, wherein at least one force amplification structure is curved or shaped to facilitate the directional force applied to an adjacent biometric sensor.

Example A-77 may include the subject matter of any of Examples A-1 to A-76, and alternatively or additionally any other example herein, wherein at least one force amplification structure is contoured to follow the shape of the underlying biometric sensor or body of the subject.

Example A-78 may include the subject matter of any of Examples A-1 to A-77, and alternatively or additionally any other example herein, wherein at least one force amplification structure includes a guidance means (e.g., a well, an aperture, a shaped valley, a depression, a boss, a magnet, or some other like structure) to improve positioning of the force amplification structure.

Example A-79 may include the subject matter of any of Examples A-1 to A-78, and alternatively or additionally any other example herein, wherein at least one force amplification structure has a shape, dimensions, materials, and other properties selected to provide a desired amount of directional pressure that will be applied to a respective biometric sensor.

Example A-80 may include the subject matter of any of Examples A-1 to A-79, and alternatively or additionally any other example herein, wherein at least one a force amplification receptacle includes a first surface proximal the body of the subject and a second surface distal the body of the subject, wherein the first and second surfaces of the force amplification receptacle at issue are formed from a same material in some cases, and formed from different materials in other cases.

Example A-81 may include the subject matter of any of Examples A-1 to A-80, and alternatively or additionally any other example herein, wherein at least one a force amplification receptacle includes a first surface proximal the body of the subject and a second surface distal the body of the subject, wherein the first surface of the force amplification receptacle (i.e., the surface closest to the body of the subject) is formed from a soft, flexible material, and the second surface of the force amplification receptacle (i.e., the surface furthest from the body of the subject) is formed from a hard, rigid material.

Example A-82 may include the subject matter of any of Examples A-1 to A-81, and alternatively or additionally any other example herein, wherein the band structure has a segmented configuration formed as a single piece of material having a generally rectangular shape and a cooperative linking mechanism.

Example A-83 may include the subject matter of any of Examples A-1 to A-82, and alternatively or additionally any other example herein, wherein the band structure has a segmented configuration formed from two or more pieces of material and any suitable number of linking mechanisms.

Example A-84 may include the subject matter of any of Examples A-1 to A-83, and alternatively or additionally any other example herein, wherein the band structure includes at least one linking mechanism, and said linking mechanism may include a single substructure or a plurality of substructures, any number of which linking substructures may have same or different configurations.

Example A-85 may include the subject matter of any of Examples A-1 to A-84, and alternatively or additionally any other example herein, wherein the band structure includes at least one linking mechanism, and said linking mechanism includes at least one of an adhesive, a hook, a catch, a loop, hook-and-loop structure, a clasp, a buckle, a hasp, a clip, a clamp, a fastener, or some other linking means.

Example A-86 may include the subject matter of any of Examples A-1 to A-85, and alternatively or additionally any other example herein, wherein the band structure is arranged as an unbroken, flexible, belt-like structure arranged with a particular width between about one half inch (0.5″) and about fifteen inches (15″).

Example A-87 may include the subject matter of any of Examples A-1 to A-86, and alternatively or additionally any other example herein, wherein the band structure is arranged as an unbroken, flexible belt-like structure arranged with a particular width of about three to four inches (3″ to 4″).

Example A-88 may include the subject matter of any of Examples A-1 to A-87, and alternatively or additionally any other example herein, wherein In the band structure is arranged in a generally cylindrical form.

Example A-89 may include the subject matter of any of Examples A-1 to A-88, and alternatively or additionally any other example herein, wherein In the band structure is flexible and configured to conform to the body portion of interest of the subject when deployed.

Example A-90 may include the subject matter of any of Examples A-1 to A-89, and alternatively or additionally any other example herein, wherein In the band structure is arranged in a generally cylindrical form with a circular top-down cross section having a diameter between about five inches (5″) and about twenty inches (20″).

Example A-91 may include the subject matter of any of Examples A-1 to A-90, and alternatively or additionally any other example herein, wherein In the band structure is arranged in a segmented configuration having a generally rectangular form.

Example A-92 may include the subject matter of any of Examples A-1 to A-91, and alternatively or additionally any other example herein, wherein In the band structure is arranged in a segmented configuration having a generally rectangular shape with a length of between about eighteen inches (18″) and about sixty inches (60″).

Example A-93 may include the subject matter of any of Examples A-1 to A-92, and alternatively or additionally any other example herein, wherein the subject is involved in activity related to baseball, hockey, golf, soccer, kickball, biking, running, tennis, racquetball, archery, hunting, work (e.g., military, construction, law enforcement, firefighting, or any other work), or some other physically-, mentally-, or emotionally-stressful activity when the biometric wearable device is deployed.

Example A-94 may include the subject matter of any of Examples A-1 to A-93, and alternatively or additionally any other example herein, wherein the biometric sensor is arranged to capture any one or more of heart-signal data, temperature data, respiration data, oxygen saturation data, hydration data, or some other physiologic data associated with the subject.

Example A-95 may include the subject matter of any of Examples A-1 to A-94, and alternatively or additionally any other example herein, wherein data collected by the biometric sensor is partially or fully processed by an onboard processor.

Example A-96 may include the subject matter of any of Examples A-1 to A-95, and alternatively or additionally any other example herein, wherein data is collected by the biometric sensor, and wherein at least one of raw data, processed data, or some combination of raw and processed data is communicated to another computing device via onboard communications circuitry.

Example A-97 may include the subject matter of any of Examples A-1 to A-96, and alternatively or additionally any other example herein, wherein the biometric sensor is arranged to process collected data with an onboard processor, and wherein such processing includes any one or more of summing or otherwise accumulating data, averaging data, identifying data above or below a determined threshold (e.g., a selected heartrate, a selected temperature, a selected oxygen saturation level, and the like), combining data, generating a particular diagnosis about the subject based on the data (e.g., the subject's health is in danger, the subject is in a weight-loss zone, the subject is in a muscle-building zone, and the like), and generating other such conclusions.

Example A-98 may include the subject matter of any of Examples A-1 to A-97, and alternatively or additionally any other example herein, wherein the biometric sensor is arranged to process collected data with an onboard processor, generate additional data with the onboard processor, communicate at least one of the collected data and generated additional data to an external computing device via communications circuitry.

Example A-99 may include the subject matter of any of Examples A-1 to A-98, and alternatively or additionally any other example herein, wherein the biometric sensor is arranged to communicate data via one or more of an electromechanical connector and a communications medium such as wire coupleable to the electromechanical connector.

Example A-100 may include the subject matter of any of Examples A-1 to A-99, and alternatively or additionally any other example herein, wherein the biometric sensor is arranged to communicate data via one or more of a wired or wireless transmitter, a wired or wireless receiver, or a wired or wireless transceiver operating in accordance with either or both of a proprietary protocol and a known protocol such as universal serial bus (USB), BLUETOOTH, or the like.

Example A-101 may include the subject matter of any of Examples A-1 to A-100, and alternatively or additionally any other example herein, wherein data communicated from a band structure is arranged to pass through a communications network, and wherein said communications network includes any one or more of a direct peer-to-peer communications network (e.g., a wire, a single conduit cable, or a cable having a multipath set of conduits, a shared communications network such as Ethernet or USB, and wherein some or all of said communications network may be a wireless communications network operating under any suitable protocol (e.g., cellular, WiFi, BLUETOOTH, and the like).

Example A-102 may include the subject matter of any of Examples A-1 to A-101, and alternatively or additionally any other example herein, wherein data communicated from a band structure through a communications network is arranged for processing by a mobile computing device, a computing server, or both a mobile computing device and a computing server.

Example A-103 may include the subject matter of any of Examples A-1 to A-102, and alternatively or additionally any other example herein, wherein data communicated from a band structure through a communications network is arranged for storage in a data repository.

Example A-104 may include the subject matter of any of Examples A-1 to A-103, and alternatively or additionally any other example herein, wherein the biometric sensor is an electrocardiogram (ECG) sensor or some other capacitive electrode.

Example A-105 may include the subject matter of any of Examples A-1 to A-104, and alternatively or additionally any other example herein, wherein the biometric sensor is formed as a thin metal electrode with a thin oxide coating.

Example A-106 may include the subject matter of any of Examples A-1 to A-105, and alternatively or additionally any other example herein, wherein the biometric sensor is formed of a flexible metal material.

Example A-107 may include the subject matter of any of Examples A-1 to A-106, and alternatively or additionally any other example herein, wherein the biometric sensor further includes at least one of signal conditioning electronic circuitry and signal capturing electronic circuitry.

Example A-108 may include the subject matter of any of Examples A-1 to A-107, and alternatively or additionally any other example herein, wherein the biometric sensor further includes electronic circuitry formed of flexible material.

Example A-109 may include the subject matter of any of Examples A-1 to A-108, and alternatively or additionally any other example herein, wherein when the biometric sensor is put into operation, changes in biological signals of the subject will electrostatically induce a charge in the biometric sensor.

Example A-110 may include the subject matter of any of Examples A-1 to A-109, and alternatively or additionally any other example herein, wherein the biometric wearable device includes multiple biometric sensors (e.g., from two to twelve or more sensors).

Example A-111 may include the subject matter of any of Examples A-1 to A-110, and alternatively or additionally any other example herein, wherein the biometric wearable device includes multiple biometric sensors, and wherein during operation, at least one electrode of one sensor is driven to a ground potential.

Example A-112 may include the subject matter of any of Examples A-1 to A-111, and alternatively or additionally any other example herein, wherein one or more biometric sensors are arranged to capture temperature data, said temperature data being associated with one or more of a subject, an environment, an object, or some other thing.

Example A-113 may include the subject matter of any of Examples A-1 to A-112, and alternatively or additionally any other example herein, wherein one or more biometric sensors are arranged to capture temperature data representative of the body of the subject.

Example A-114 may include the subject matter of any of Examples A-1 to A-113, and alternatively or additionally any other example herein, wherein one or more biometric sensors are arranged to capture temperature data, said one or more biometric sensors including metal-based (e.g., platinum, nickel, copper, and the like) conductive elements that change resistance under calibratable or otherwise know conditions.

Example A-115 may include the subject matter of any of Examples A-1 to A-114, and alternatively or additionally any other example herein, wherein one or more biometric sensors are arranged to capture temperature data via one or more metal-based conductive elements having a current applied to the element and surrounding circuitry and a voltage measured across an active element of the sensor, wherein the measured voltage may be used to generate one or more static, dynamic, or static and dynamic temperature values.

Example A-116 may include the subject matter of any of Examples A-1 to A-115, and alternatively or additionally any other example herein, wherein one or more biometric sensors are configured to generate respiration data.

Example A-117 may include the subject matter of any of Examples A-1 to A-116, and alternatively or additionally any other example herein, wherein one or more biometric sensors are configured to produce data representative of a respiration signal, said respiration signal being a relative count or measure of expansion of the subject's abdomen or thorax.

Example A-118 may include the subject matter of any of Examples A-1 to A-117, and alternatively or additionally any other example herein, wherein one or more biometric sensors are arranged as a sensitive girth sensor that detects abdominal expansion and contraction (i.e., displacement) and that generates respiration waveform and amplitude data representative of the subject's respiratory cycles, patterns, or cycles and patterns.

Example A-119 may include the subject matter of any of Examples A-1 to A-118, and alternatively or additionally any other example herein, wherein one or more biometric sensors are arranged to collect data representative of a subject's peripheral oxygen saturation (i.e., how much of the hemoglobin in blood is carrying oxygen (SpO2)).

Example A-120 may include the subject matter of any of Examples A-1 to A-119, and alternatively or additionally any other example herein, wherein one or more biometric sensors are arranged as oximeters, pulse oximeters, or some other like term.

Example A-121 may include the subject matter of any of Examples A-1 to A-120, and alternatively or additionally any other example herein, wherein one or more biometric sensors include a light source and a light detector.

Example A-122 may include the subject matter of any of Examples A-1 to A-121, and alternatively or additionally any other example herein, wherein one or more biometric sensors include a light source and a light detector, wherein the light source is arranged to generate certain light (e.g., red light and infrared light) and direct such light through biological material of the subject (e.g., a finger or some other part of a subject's anatomy), and wherein the light detector is arranged to detect an amount of emitted light that passes through the portion of the subject's body that is associated with the sensor.

Example A-123 may include the subject matter of any of Examples A-1 to A-122, and alternatively or additionally any other example herein, wherein one or more biometric sensors are arranged to generate hydration data associated with the subject.

Example A-124 may include the subject matter of any of Examples A-1 to A-123, and alternatively or additionally any other example herein, wherein one or more biometric sensors are arranged to generate hydration data associated with the subject, said hydration data representing one or more of a rate of perspiration of the subject, a state of hydration of the subject, a core body temperature of the subject, a loss of electrolytes suffered by the subject, or some other hydration data.

Example A-125 may include the subject matter of any of Examples A-1 to A-124, and alternatively or additionally any other example herein, wherein one or more biometric sensors are arranged to collect and analyze bodily fluid (e.g., perspiration) of the subject, apply various excitation signals, and based on corresponding response signals, generate various impedance values.

Example A-126 may include the subject matter of any of Examples A-1 to A-125, and alternatively or additionally any other example herein, wherein one or more biometric sensors are arranged to collect and analyze bodily fluid, generate various impedance values, and use said impedance values to generate the hydration data.

Example A-127 may include the subject matter of any of Examples A-1 to A-126, and alternatively or additionally any other example herein, wherein a band positioned at the heart-active region of the garment includes a multilayer superstructure having two layers or three layers, and each layer is formed of a fabric, a plastic, or some other material.

Example A-128 may include the subject matter of any of Examples A-1 to A-127, and alternatively or additionally any other example herein, wherein a band positioned at the heart-active region of the garment includes a multilayer superstructure having two layers or three layers that are welded, stitched, or welded and stitched to each other.

Example A-129 may include the subject matter of any of Examples A-1 to A-128, and alternatively or additionally any other example herein, wherein a band formed in the garment is arranged to provide two or more different levels of compression in different zones defined on the band, wherein each zonal region is formed as a sequence of apertures, thin areas, or some other structure.

Example A-130 may include the subject matter of any of Examples A-1 to A-129, and alternatively or additionally any other example herein, wherein a band formed in the garment has a first zonal compression region having a first compression, a second zonal compression region having a second compression, and third zonal compression region having a third compression, wherein the first zonal compression region has a first higher recoverable tensile strain under stress than the second zonal compression region, and wherein the second zonal compression region has a second higher recoverable tensile strain under stress than the third zonal compression region.

The various embodiments described above can be combined to provide further embodiments. Various features of the embodiments are optional, and, features of one embodiment may be suitably combined with other embodiments. Aspects of the embodiments can be modified, if necessary to employ concepts of the various patents, applications, and publications to provide yet further embodiments.

This application claims the priority benefit of U.S. Provisional Patent Application No. 63/263,858, filed Nov. 10, 2021, which application is hereby incorporated by reference in its entirety.

In the description herein, specific details are set forth in order to provide a thorough understanding of the various example embodiments. It should be appreciated that various modifications to the embodiments will be readily apparent to those skilled in the art, and the generic principles defined herein may be applied to other embodiments and applications without departing from the spirit and scope of the disclosure. Moreover, in the following description, numerous details are set forth for the purpose of explanation. However, one of ordinary skill in the art should understand that embodiments may be practiced without the use of these specific details. In other instances, well-known structures and processes are not shown or described in order to avoid obscuring the description with unnecessary detail. Thus, the present disclosure is not intended to be limited to the embodiments shown but is instead to be accorded the widest scope consistent with the principles and features disclosed herein. Hence, these and other changes can be made to the embodiments in light of the above-detailed description. In general, in the following claims, the terms used should not be construed to limit the claims to the specific embodiments disclosed in the specification but should be construed to include all possible embodiments along with the full scope of equivalents to which such claims are entitled. Accordingly, the claims are not limited by the disclosure.

1. A biometric wearable device, comprising:
a garment arranged for wear about a torso of a subject, the garment including:
a first opening for substantially encircling a neck area of the subject that is wearing the garment;
a second opening for substantially encircling a right extremity of the subject;
a third opening for substantially encircling a left extremity of the subject;
a fourth opening for substantially encircling a lower portion of the torso of the subject; and
a heart-active region which, when the subject is wearing the garment, is proximate an area of the torso of the subject where heart-caused signals are detectable;
a biometric sensor arranged to detect the heart-caused signals of the subject when the subject is wearing the garment;
a band positioned at the heart-active region of the garment and arranged to contain the biometric sensor; and
at least one force amplification structure positionable in a force amplification receptacle of the band, wherein the at least one force amplification structure is arranged to provide increased directional pressure to the biometric sensor.

2. The device of claim 1 wherein the band is arranged to position an active portion of the at least one biometric sensor in direct contact with the torso of the subject.

3. The device of claim 1 wherein the biometric sensor includes at least two electrodes.

4. The device of claim 1 wherein the band is at least ten inches (10 in.) long.

5. The device of claim 1 wherein the band is a continuous band having at diameter of between about eight to fifteen inches (8 in. to 15 in.).

6. The device of claim 1 wherein the band is between about one to four inches (1 in. to 4 in.) wide.

7. The device of claim 1 wherein the band is between about one to five hundred mils (0.001 in. to 0.5 in.) thick.

8. The device of claim 1 wherein the band is formed from a material having elastic properties.

9. The device of claim 1 wherein the band is formed from a first material having stronger elastic properties than a second material used to form the garment.

10. The device of claim 1 wherein the band exposes at least one electromechanical structure arranged to pass signal information associated with the at least one biometric sensor.

11. The device of claim 8 wherein the at least one electromechanical structure is a wire or an electrical connector.

12. The device of claim 1 wherein the garment is a shirt.

13. The device of claim 1 wherein the subject is a human being.

14. The device of claim 1 wherein the subject is a non-human mammal.

15. The device of claim 1 wherein the biometric sensor is one of a plurality of biometric sensors integrated in the band.

16. A method, comprising:
providing a garment with a band, the band having a biometric sensor, the band further having a force amplification receptacle;
positioning at least one force amplification structure in the force amplification receptacle of the band, wherein the at least one force amplification structure is arranged to provide increased directional pressure to the biometric sensor; and
detecting, with the biometric sensor, at least one heart-caused signal of a subject wearing the garment.

17. The method of claim 16, comprising:
detecting, with the biometric sensor, a plurality of heart-caused signals of the subject wearing the garment over a determined period of time.

18. The method of claim 16, comprising:
communicating the at least one heart-caused signal to a remote computing device.

19. The method of claim 16, comprising:
improving quality of the detected at least one heart-caused signal by adding at least one additional force amplification structure to the force amplification receptacle or removing one of the at least one force amplification structures from the force amplification receptacle.

20. A system, comprising:
a garment arranged for wear by a subject, the garment including a heart-active region which, when the garment is worn by the subject, is proximate an area of the subject where heart-caused signals are detectable;
a biometric sensor arranged to detect the heart-caused signals of the subject when the subject is wearing the garment;
a band positioned at least in part at the heart-active region of the garment and arranged to contain the biometric sensor;
at least one force amplification structure positioned in a force amplification receptacle of the band, wherein the at least one force amplification structure is arranged to provide increased directional pressure to the biometric sensor; and
at least one remote computing device arranged to receive information communicated from the biometric sensor, said information representative of the detected heart-caused signals.
